(19) United States
(12) Patent Application Publication
(10) Pub. No.: US 2023/0041071 A1
(43) Pub. Date: Feb. 9, 2023

(54) ANTIBODIES AGAINST AREG AND ITS USE

(71) Applicant: National Institute of Biological Sciences, Beijing, Beijing (CN)

(72) Inventors: Jianhua Sui, Beijing (CN); Ximing Liu, Beijing (CN); Nan Tang, Beijing (CN); Huijuan Wu, Beijing (CN)

(21) Appl. No.: 17/935,400

(22) Filed: Sep. 26, 2022

Related U.S. Application Data
(63) Continuation-in-part of application No. PCT/CN2021/082027, filed on Mar. 22, 2021.

(30) Foreign Application Priority Data
Mar. 27, 2020 (CN) PCT/CN2020/081785

(51) Int. Cl. C07K 16/22; A61P 11/00

(52) U.S. Cl. CPC C07K 16/22; A61P 11/00; C07K 2317/24; C07K 2317/54; C07K 2317/92; C07K 2317/565

(57) ABSTRACT

Provided are anti-AREG antibodies or immunoreactive fragments thereof for the treatment, diagnosis or prophylaxis of fibrotic diseases, including but not limited to renal fibrosis, hepatic fibrosis, pulmonary fibrosis, in particular, IPF. Polynucleotides or nucleic acid molecules encoding the antibodies, expression vectors, host cells and methods for making the antibodies are also provided. The anti-AREG antibodies specifically bind to AREG and block the function of AREG, through binding residues that locate in the EGF like domain.

### CROSS REFERENCE

The application claims benefit and is a continuation-in-part application of PCT Application No. PCT/CN2021/082027, filed Mar. 22, 2021, which claims benefit of PCT Application No. PCT/CN2020/081785, filed on Mar. 27, 2020. The above-identified application are incorporated herein by reference in their entireties.

### REFERENCE TO AN ELECTRONIC SEQUENCE LISTING

The contents of the electronic sequence listing (109307-1348935-000400US_Seq-Listing; Size: 180 KB; and Date of Creation: Sep. 26, 2022) is herein incorporated by reference in its entirety.

### INTRODUCTION

Fibrosis, the thickening and scarring of connective tissue that can result from injury, is characterized by the excessive proliferation of fibroblast cells and the accumulation of extracellular matrix (ECM) components. This disorder, which is commonly observed in organs including lungs, livers, and kidneys, among many others, causes disrupted tissue architecture and leads to major impairments in organ function.

Pulmonary fibrosis (PF) is a lung disease that occurs when healthy lung tissue is replaced by excess extracellular matrix. The alveolar structure of PF lungs is destroyed, and this results in reduced lung compliance, impaired gas exchange, and ultimately respiratory failure and death. The common feature of pulmonary fibrosis is excessive proliferation of fibroblasts around the air sacs of the lungs (alveoli) (Barkauskas and Noble, 2014). The most common type of pulmonary fibrosis is idiopathic pulmonary fibrosis (IPF). IPF is an interstitial lung disease with unknown cause and serious progressive loss of lung function. It most commonly occurs in elderly people aged 50 to 70. IPF is a fatal disease with a median survival time of only 2-4 years from diagnosis (Steele and Schwartz, 2013), and can ultimately lead to respiratory failure. The pathogenesis of pulmonary fibrosis has been an unsolved mystery, and the clinical treatment is very limited. There are currently only two commercially available FDA-approved drugs, Nintedanib and Pirfenidone, for the treatment of IPF. However, both drugs can only improve the rate of decline in forced vital capacity within one year; neither of them can significantly increase patient survival.

The following lists the prior art of anti-AREG antibodies:

U.S. patent application Ser. No. 10/774,076 relates to AREG antibodies and their use to treat cancer and psoriasis. The claimed antibody is a humanized PAR34;

PCT application No. PCT/GB2009/050389 relates to antibodies cross-reacting with both AREG and HBEGF. The antibodies may be used in methods of treatment of cancer and diseases associated with angiogenesis. The claimed antibody is 2F7; and

U.S. patent application Ser. No. 15/271,515 relates to AREG antibodies and their use to treat cancer. The claimed antibodies are AR30, AR37 and AR558. Among them, AR558 showed the best anti-tumor activity in a xenograft mouse tumor model. All these three antibodies are murine antibodies, rather than humanized antibodies.

### SUMMARY OF THE INVENTION

In the prior art, no affirmatory reports on the key drug target for pulmonary fibrosis, in particular, idiopathic pulmonary fibrosis (IPF), especially, AREG signaling in AT2 cells of the lung, were published. The inventors of the present invention establish an unique connection between the AREG signaling in AT2 cells of the lung and the development of pulmonary fibrosis, in particular, IPF, and find that AREG signaling in AT2 cells of the lung can be used as the key drug target for pulmonary fibrosis, in particular, IPF. Specifically, AREG is not detected in AT2 cells of normal control lungs, but is detected in AT2 cells of all IPF specimens.

Furthermore, the inventors of the present invention construct an animal model of IPF, wherein Cdc42 gene in AT2 cells is knocked out. AREG can't be detected in AT2 cells of control lungs, but can be detected in AT2 cells of Cdc42 AT2 null lungs. This is the first animal model that can highly mimic the pathogenesis and progression of IPF. Using this animal model, we identified that AREG is a key therapeutic target for pulmonary fibrosis.

Based on the above knowledge, the inventors of the present invention prepare, screen and obtain antibodies against AREG for treatment of renal fibrosis, hepatic fibrosis, pulmonary fibrosis, in particular, IPF.

The present invention provides anti-AREG antibodies or immunoreactive fragments thereof for the treatment, diagnosis or prophylaxis of fibrotic diseases, including but not limited to renal fibrosis, hepatic fibrosis, pulmonary fibrosis, in particular, IPF. Polynucleotides or nucleic acid molecules encoding the antibodies, expression vectors, host cells and methods for making the antibodies are also provided. Pharmaceutical compositions comprising the antibody molecules are also provided. The anti-AREG antibodies of the present invention specifically bind to AREG and block the function of AREG, through binding residues that locate in the EGF like domain. The anti-AREG antibodies disclosed herein can be used to treat, prevent and/or diagnose fibrotic diseases including but not limited to renal fibrosis, hepatic fibrosis, pulmonary fibrosis, in particular, IPF.

In one aspect, the present invention provides an isolated anti-AREG antibody or fragment thereof having the ability of inhibiting fibrosis. Preferably, the fibrosis is renal fibrosis, hepatic fibrosis, pulmonary fibrosis, in particular, IPF.

In some embodiments, the anti-AREG antibody or fragment thereof according to the present invention is capable of binding AREG.

In some embodiments, the anti-AREG antibody or fragment thereof according to the present invention binds to both human AREG (hAREG) and mouse AREG (mAREG).

In some embodiments, the anti-AREG antibody or fragment thereof according to the present invention merely binds to human AREG (hAREG), and fails to bind mouse AREG (mAREG).

In some embodiments, the anti-AREG antibody or fragment thereof according to the present invention is a human anti-AREG antibody, or a murine anti-AREG antibody, or a humanized anti-AREG antibody, or a chimeric anti-AREG antibody.

In some embodiments, the anti-AREG antibody or fragment thereof according to the present invention binds to AREG with high affinity, with a dissociation constant (KD) of less than about 10 nM, e.g., less than 1 nM, 0.1 nM, or 0.01 nM, for example, in the range of 1×10⁻⁸-1×10⁻¹¹, preferably, in the range of 1×10⁻⁹-1×10⁻¹¹.

In some embodiments, the anti-AREG antibody or fragment thereof according to the present invention is capable of binding to soluble forms of AREG. Preferably, the anti-AREG antibody is capable of binding to EGF-like domain of soluble forms of AREG.

In some embodiments, the anti-AREG antibody or fragment thereof according to the present invention is capable of binding to residues 101-184 of the human pro-AREG. The amino acid sequence of human pro-AREG is shown in SEQ ID NO: 135.

In some embodiments, the anti-AREG antibody is capable of binding to C-terminus within EGF-like domain of soluble forms of AREG.

In some embodiments, the anti-AREG antibody or fragment thereof according to the present invention is capable of binding to residues 171-184 of the human pro-AREG.

In some embodiments, the anti-AREG antibody or fragment thereof according to the present invention is capable of binding to residues 94-177 of the murine pro-AREG.

In some embodiments, the anti-AREG antibody or fragment thereof according to the present invention is capable of binding to EGF-like domain, residues 135-177 of the murine pro-AREG.

In some embodiments, the anti-AREG antibody or fragment thereof according to the present invention is capable of binding, e.g., at least one, two, three, four or five amino acids within residues 101-184 of human pro-AREG shown by any one of SEQ ID NOs: 123-132, preferably, within residues 142-184 of human pro-AREG shown by any one of SEQ ID NOs: 123-132.

In some embodiments, the anti-AREG antibody or fragment thereof according to the present invention is capable of interacting with Glu149 and/or His164 of human pro-AREG.

In some embodiments, the anti-AREG antibody or fragment thereof according to the present invention is capable of binding, e.g., at least one, two, three, four or five amino acids within residues 94-177 of murine pro-AREG, preferably, within residues 137-177 of murine pro-AREG.

In some embodiments, the anti-AREG antibody or fragment thereof according to the present invention is an antibody fragment that binds to soluble forms of AREG.

In some embodiments, the anti-AREG antibody or fragment thereof according to the present invention is Fab fragment or F(ab)₂ fragment.

In some embodiments, the anti-AREG antibody or fragment thereof according to the present invention comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:

HCDR1, HCDR2, and HCDR3 are selected from the group consisting of: (1) HCDR1 shown by SEQ ID NO: 1, HCDR2 shown by SEQ ID NO: 2, HCDR3 shown by SEQ ID NO: 3; (2) HCDR1 shown by SEQ ID NO: 1, HCDR2 shown by SEQ ID NO: 2, HCDR3 shown by SEQ ID NO: 4; (3) HCDR1 shown by SEQ ID NO: 5, HCDR2 shown by SEQ ID NO: 2, HCDR3 shown by SEQ ID NO: 6; (4) HCDR1 shown by SEQ ID NO: 7, HCDR2 shown by SEQ ID NO: 8, HCDR3 shown by SEQ ID NO: 9; (5) HCDR1 shown by SEQ ID NO: 7, HCDR2 shown by SEQ ID NO: 10, HCDR3 shown by SEQ ID NO: 9; (6) HCDR1 shown by SEQ ID NO: 7, HCDR2 shown by SEQ ID NO: 8, HCDR3 shown by SEQ ID NO: 11; (7) HCDR1 shown by SEQ ID NO: 7, HCDR2 shown by SEQ ID NO: 8, HCDR3 shown by SEQ ID NO: 12; (8) HCDR1 shown by SEQ ID NO: 1, HCDR2 shown by SEQ ID NO: 13, HCDR3 shown by SEQ ID NO: 14; (9) HCDR1 shown by SEQ ID NO: 1, HCDR2 shown by SEQ ID NO: 15, HCDR3 shown by SEQ ID NO: 16; (10) HCDR1 shown by SEQ ID NO: 17, HCDR2 shown by SEQ ID NO: 18, HCDR3 shown by SEQ ID NO: 19; (11) HCDR1 shown by SEQ ID NO: 17, HCDR2 shown by SEQ ID NO: 18, HCDR3 shown by SEQ ID NO: 20; (12) HCDR1 shown by SEQ ID NO: 1, HCDR2 shown by SEQ ID NO: 13, HCDR3 shown by SEQ ID NO: 136; and (13) HCDR1, HCDR2, HCDR3 as shown in (1)-(12), but at least one of which includes one, two, three, four or five amino acids addition, deletion, conservative amino acid substitution or the combinations thereof; and
LCDR1, LCDR2, and LCDR3 are selected from the group consisting of:
(1) LCDR1 shown by SEQ ID NO: 21, LCDR2 shown by SEQ ID NO: 22, LCDR3 shown by SEQ ID NO: 23; (2) LCDR1 shown by SEQ ID NO: 21, LCDR2 shown by SEQ ID NO: 22, LCDR3 shown by SEQ ID NO: 24; (3) LCDR1 shown by SEQ ID NO: 25, LCDR2 shown by SEQ ID NO: 26, LCDR3 shown by SEQ ID NO: 27; (4) LCDR1 shown by SEQ ID NO: 28, LCDR2 shown by SEQ ID NO: 29, LCDR3 shown by SEQ ID NO: 30; (5) LCDR1 shown by SEQ ID NO: 31, LCDR2 shown by SEQ ID NO: 32, LCDR3 shown by SEQ ID NO: 30; (6) LCDR1 shown by SEQ ID NO: 33, LCDR2 shown by SEQ ID NO: 34, LCDR3 shown by SEQ ID NO: 30; (7) LCDR1 shown by SEQ ID NO: 35, LCDR2 shown by SEQ ID NO: 34, LCDR3 shown by SEQ ID NO: 30; (8) LCDR1 shown by SEQ ID NO: 36, LCDR2 shown by SEQ ID NO: 37, LCDR3 shown by SEQ ID NO: 38; (9) LCDR1 shown by SEQ ID NO: 39, LCDR2 shown by SEQ ID NO: 40, LCDR3 shown by SEQ ID NO: 38; (10) LCDR1 shown by SEQ ID NO: 41, LCDR2 shown by SEQ ID NO: 42, LCDR3 shown by SEQ ID NO: 38; (11) LCDR1 shown by SEQ ID NO: 43, LCDR2 shown by SEQ ID NO: 44, LCDR3 shown by SEQ ID NO: 38; (12) LCDR1 shown by SEQ ID NO: 39, LCDR2 shown by SEQ ID NO: 40, LCDR3 shown by SEQ ID NO: 38; (13) LCDR1 shown by SEQ ID NO: 45, LCDR2 shown by SEQ ID NO: 42, LCDR3 shown by SEQ ID NO: 46; (14) LCDR1SEQ ID NO: 47, LCDR2 shown by SEQ ID NO: 44, LCDR3 shown by SEQ ID NO: 46; (15) LCDR1 shown by SEQ ID NO: 48, LCDR2 shown by SEQ ID NO: 37, LCDR3 shown by SEQ ID NO: 49; (16) LCDR1 shown by SEQ ID NO: 50, LCDR2 shown by SEQ ID NO: 40, LCDR3 shown by SEQ ID NO: 51; (17) LCDR1 shown by SEQ ID NO: 50, LCDR2 shown by SEQ ID NO: 40, LCDR3 shown by SEQ ID NO: 52; (18) LCDR1 shown by SEQ ID NO: 50, LCDR2 shown by SEQ ID NO: 40, LCDR3 shown by SEQ ID NO: 53; (19) LCDR1 shown by SEQ ID NO: 54, LCDR2 shown by SEQ ID NO: 42, LCDR3 shown by SEQ ID NO: 55; (20) LCDR1 shown by SEQ ID NO: 56, LCDR2 shown by SEQ ID NO: 44, LCDR3 shown by SEQ ID NO: 55; and (21) LCDR1, LCDR2, LCDR3 as shown in (1)-(20), but at least one of which includes one, two, three, four or five amino acids addition, deletion, conservative amino acid substitution or the combinations thereof.

In one embodiment, the anti-AREG antibody or fragment thereof according to the present invention comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:

HCDR1, HCDR2, HCDR3, LCDR1, LCDR2, and LCDR3 are selected from the group consisting of: (1) HCDR1 shown by SEQ ID NO: 1, HCDR2 shown by SEQ ID NO: 2, HCDR3 shown by SEQ ID NO: 3, LCDR1 shown by SEQ ID NO: 21, LCDR2 shown by SEQ ID NO: 22, LCDR3 shown by SEQ ID NO: 23; (2) HCDR1 shown by SEQ ID NO: 1, HCDR2 shown by SEQ ID NO: 2, HCDR3 shown by SEQ ID NO: 4, LCDR1 shown by SEQ ID NO: 21, LCDR2 shown by SEQ ID NO: 22, LCDR3 shown by SEQ ID NO: 24; (3) HCDR1 shown by SEQ ID NO: 5, HCDR2 shown by SEQ ID NO: 2, HCDR3 shown by SEQ ID NO: 6, LCDR1 shown by SEQ ID NO: 25, LCDR2 shown by SEQ ID NO: 26, LCDR3 shown by SEQ ID NO: 27; (4) HCDR1 shown by SEQ ID NO: 7, HCDR2 shown by SEQ ID NO: 8, HCDR3 shown by SEQ ID NO: 9, LCDR1 shown by SEQ ID NO: 28, LCDR2 shown by SEQ ID NO: 29, LCDR3 shown by SEQ ID NO: 30; (5) HCDR1 shown by SEQ ID NO: 7, HCDR2 shown by SEQ ID NO: 10, HCDR3 shown by SEQ ID NO: 9, LCDR1 shown by SEQ ID NO: 31, LCDR2 shown by SEQ ID NO: 32, LCDR3 shown by SEQ ID NO: 30; (6) HCDR1 shown by SEQ ID NO: 7, HCDR2 shown by SEQ ID NO: 8, HCDR3 shown by SEQ ID NO: 11, LCDR1 shown by SEQ ID NO: 33, LCDR2 shown by SEQ ID NO: 34, LCDR3 shown by SEQ ID NO: 30; (7) HCDR1 shown by SEQ ID NO: 7, HCDR2 shown by SEQ ID NO: 8, HCDR3 shown by SEQ ID NO: 12, LCDR1 shown by SEQ ID NO: 35, LCDR2 shown by SEQ ID NO: 34, LCDR3 shown by SEQ ID NO: 30; (8) HCDR1 shown by SEQ ID NO: 1, HCDR2 shown by SEQ ID NO: 13, HCDR3 shown by SEQ ID NO: 14, LCDR1 shown by SEQ ID NO: 36, LCDR2 shown by SEQ ID NO: 37, LCDR3 shown by SEQ ID NO: 38; (9) HCDR1 shown by SEQ ID NO: 1, HCDR2 shown by SEQ ID NO: 13, HCDR3 shown by SEQ ID NO: 136, LCDR1 shown by SEQ ID NO: 39, LCDR2 shown by SEQ ID NO: 40, LCDR3 shown by SEQ ID NO: 38; (10) HCDR1 shown by SEQ ID NO: 1, HCDR2 shown by SEQ ID NO: 13, HCDR3 shown by SEQ ID NO: 136, LCDR1 shown by SEQ ID NO: 41, LCDR2 shown by SEQ ID NO: 42, LCDR3 shown by SEQ ID NO: 38; (11) HCDR1 shown by SEQ ID NO: 1, HCDR2 shown by SEQ ID NO: 13, HCDR3 shown by SEQ ID NO: 136, LCDR1 shown by SEQ ID NO: 43, LCDR2 shown by SEQ ID NO: 44, LCDR3 shown by SEQ ID NO: 38; (12) HCDR1 shown by SEQ ID NO: 1, HCDR2 shown by SEQ ID NO: 15, HCDR3 shown by SEQ ID NO: 16, LCDR1 shown by SEQ ID NO: 39, LCDR2 shown by SEQ ID NO: 40, LCDR3 shown by SEQ ID NO: 38; (13) HCDR1 shown by SEQ ID NO: 1, HCDR2 shown by SEQ ID NO: 15, HCDR3 shown by SEQ ID NO: 16, LCDR1 shown by SEQ ID NO: 45, LCDR2 shown by SEQ ID NO: 42, LCDR3 shown by SEQ ID NO: 46; (14) HCDR1 shown by SEQ ID NO: 1, HCDR2 shown by SEQ ID NO: 15, HCDR3 shown by SEQ ID NO: 16, LCDR1 shown by SEQ ID NO: 47, LCDR2 shown by SEQ ID NO: 44, LCDR3 SHOWN BY SEQ ID NO: 46; (15) HCDR1 shown by SEQ ID NO: 17, HCDR2 shown by SEQ ID NO: 18, HCDR3 shown by SEQ ID NO: 19, LCDR1 shown by SEQ ID NO: 48, LCDR2 shown by SEQ ID NO: 37, LCDR3 shown by SEQ ID NO: 49; (16) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 51; (17) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 52; (18) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 53; (19) HCDR1 shown by SEQ ID NO: 17, HCDR2 shown by SEQ ID NO: 18, HCDR3 shown by SEQ ID NO: 20, LCDR1 shown by SEQ ID NO: 54, LCDR2 shown by SEQ ID NO: 42, LCDR3 shown by SEQ ID NO: 55; (20) HCDR1 shown by SEQ ID NO: 17, HCDR2 shown by SEQ ID NO: 18, HCDR3 shown by SEQ ID NO: 20, LCDR1 shown by SEQ ID NO: 56, LCDR2 shown by SEQ ID NO: 44, LCDR3 shown by SEQ ID NO: 55; and (21) HCDR1, HCDR2, HCDR3, LCDR1, LCDR2, LCDR3 as shown in (1)-(20), but at least one of which includes one, two, three, four or five amino acids addition, deletion, conservative amino acid substitution or the combinations thereof.

Preferably, the anti-AREG antibody or fragment thereof according to the present invention comprises HCDR1, HCDR2, and HCDR3 selected from the group consisting of:

(1) HCDR1 shown by SEQ ID NO: 5, HCDR2 shown by SEQ ID NO: 2, HCDR3 shown by SEQ ID NO: 6;
(2) HCDR1 shown by SEQ ID NO: 1, HCDR2 shown by SEQ ID NO: 2, HCDR3 shown by SEQ ID NO: 4; and
(3) HCDR1 shown by SEQ ID NO: 7, HCDR2 shown by SEQ ID NO: 10, HCDR3 shown by SEQ ID NO: 9; and LCDR1, LCDR2, and LCDR3 selected from the group consisting of:
(1) LCDR1 shown by SEQ ID NO: 25, LCDR2 shown by SEQ ID NO: 26, LCDR3 shown by SEQ ID NO: 27;
(2) LCDR1 shown by SEQ ID NO: 21, LCDR2 shown by SEQ ID NO: 22, LCDR3 shown by SEQ ID NO: 24; and
(3) LCDR1 shown by SEQ ID NO: 31, LCDR2 shown by SEQ ID NO: 32, LCDR3 shown by SEQ ID NO: 30.

Preferably, the anti-AREG antibody or fragment thereof according to the present invention comprises HCDR1, HCDR2, HCDR3, LCDR1, LCDR2, and LCDR3 selected from the group consisting of:

(1) HCDR1 shown by SEQ ID NO: 5, HCDR2 shown by SEQ ID NO: 2, HCDR3 shown by SEQ ID NO: 6, LCDR1 shown by SEQ ID NO: 25, LCDR2 shown by SEQ ID NO: 26, LCDR3 shown by SEQ ID NO: 27;
(2) HCDR1 shown by SEQ ID NO: 1, HCDR2 shown by SEQ ID NO: 2, HCDR3 shown by SEQ ID NO: 4, LCDR1 shown by SEQ ID NO: 21, LCDR2 shown by SEQ ID NO: 22, LCDR3 shown by SEQ ID NO: 24; and
(3) HCDR1 shown by SEQ ID NO: 7, HCDR2 shown by SEQ ID NO: 10, HCDR3 shown by SEQ ID NO: 9, LCDR1 shown by SEQ ID NO: 31, LCDR2 shown by SEQ ID NO: 32, LCDR3 shown by SEQ ID NO: 30.

In some embodiments, the anti-AREG antibody or fragment thereof according to the present invention comprises a heavy chain variable region, and a light chain variable region, wherein the heavy chain variable region has the amino acid sequence selected from the group consisting of SEQ ID NOs: 57-69, and an amino acid sequence having at least 95% sequence identity to any one of SEQ ID NOs: 57-69, and retaining the activity of epitope-binding, wherein the light chain variable region has the amino acid sequence selected from the group consisting of SEQ ID NOs: 70-89, and an amino acid sequence having at least 95% sequence identity to any one of SEQ ID NOs: 70-89, and retaining the activity of epitope-binding.

In some embodiments, the anti-AREG antibody or fragment thereof according to the present invention comprises a heavy chain variable region, and a light chain variable region, wherein the heavy chain variable region and the light chain variable region have the amino acid sequences selected from the group consisting of (1) SEQ ID NO: 57 and SEQ ID NO: 70; (2) SEQ ID NO: 58 and SEQ ID NO: 71; (3) SEQ ID NO: 59 and SEQ ID NO: 72; (4) SEQ ID NO: 60 and SEQ ID NO: 73; (5) SEQ ID NO: 61 and SEQ ID NO: 74; (6) SEQ ID NO: 62 and SEQ ID NO: 75; (7) SEQ ID NO: 63 and SEQ ID NO: 76; (8) SEQ ID NO: 64 and SEQ ID NO: 77; (9) SEQ ID NO: 65 and SEQ ID NO: 78; (10) SEQ ID NO: 66 and SEQ ID NO: 79; (11) SEQ ID NO: 66 and SEQ ID NO: 80; (12) SEQ ID NO: 66 and SEQ ID NO: 81; (13) SEQ ID NO: 67 and SEQ ID NO: 79; (14) SEQ ID NO: 67 and SEQ ID NO: 82; (15) SEQ ID NO: 67 and SEQ ID NO: 83; (16) SEQ ID NO: 68 and SEQ ID NO: 84; (17) SEQ ID NO: 69 and SEQ ID NO: 85; (18) SEQ ID NO: 69 and SEQ ID NO: 86; (19) SEQ ID NO: 69 and SEQ ID NO: 87; (20) SEQ ID NO: 69 and SEQ ID NO: 88; (21) SEQ ID NO: 69 and SEQ ID NO: 89; and (22) two amino acid sequences having at least 95% sequence identity to any one of (1)-(21) respectively, and retaining the activity of epitope-binding.

Preferably, the anti-AREG antibody or fragment thereof according to the present invention comprises a heavy chain variable region, and a light chain variable region, wherein the heavy chain variable region has the amino acid sequence selected from the group consisting of SEQ ID NO: 59, SEQ ID NO: 58 and SEQ ID NO: 62, and the light chain variable region has the amino acid sequence selected from the group consisting of SEQ ID NO:72, SEQ ID NO: 71 and SEQ ID NO: 75.

Preferably, the anti-AREG antibody or fragment thereof according to the present invention comprises a heavy chain variable region, and a light chain variable region, wherein the heavy chain variable region and the light chain variable region have the amino acid sequences selected from the group consisting of: (1) SEQ ID NO: 59 and SEQ ID NO: 72; (2) SEQ ID NO: 58 and SEQ ID NO: 71); and (3) SEQ ID NO: 62, and SEQ ID NO: 75.

In some embodiments, the anti-AREG antibody or fragment thereof according to the present invention is an isotype of IgG, IgM, IgA, IgE or IgD. In some embodiments, the anti-AREG antibody or fragment thereof according to the present invention is an isotype of IgG1, IgG2, IgG3, or IgG4.

In some embodiments, the antibody of the present invention is human monoclonal antibody (mAb), murine mAb, humanized mAb, or chimeric mAb.

Preferably, the human monoclonal antibody (mAb) of the present invention has the heavy chain region comprising at least two of the three CDRs shown by SEQ ID NOs: 1-3, and/or the light chain region comprising at least two of the three CDRs shown by SEQ ID NOs: 21-23.

Preferably, the human monoclonal antibody (mAb) of the present invention has the heavy chain region comprising at least two of the three CDRs shown by SEQ ID NOs: 1, 2 and 4, and/or the light chain region comprising at least two of the three CDRs shown by SEQ ID NOs: 21, 22 and 24.

Preferably, the human monoclonal antibody (mAb) of the present invention has the heavy chain region comprising at least two of the three CDRs shown by SEQ ID NOs: 5, 2 and 6, and/or the light chain region comprising at least two of the three CDRs shown by SEQ ID NOs: 25-27.

Preferably, the murine monoclonal antibody (mAb) of the present invention has the heavy chain region comprising at least two of the three CDRs shown by SEQ ID NOs: 7-9, and/or the light chain region comprising at least two of the three CDRs shown by SEQ ID NOs: 28-30.

Preferably, the murine monoclonal antibody (mAb) of the present invention has the heavy chain region comprising at least two of the three CDRs shown by SEQ ID NOs: 1, 13 and 14, and/or the light chain region comprising at least two of the three CDRs shown by SEQ ID NOs: 36-38.

Preferably, the murine monoclonal antibody (mAb) of the present invention has the heavy chain region comprising at least two of the three CDRs shown by SEQ ID NOs: 17-19, and/or the light chain region comprising at least two of the three CDRs shown by SEQ ID NOs: 48, 37 and 49.

Preferably, the humanized monoclonal antibody (mAb) of the present invention has the heavy chain region comprising at least two of the three CDRs shown by SEQ ID NOs: 7-9, and/or the light chain region comprising at least two of the three CDRs shown by SEQ ID NOs: 28-30.

Preferably, the humanized monoclonal antibody (mAb) of the present invention has the heavy chain region comprising at least two of the three CDRs shown by SEQ ID NOs: 7, 10 and 9, and/or the light chain region comprising at least two of the three CDRs shown by SEQ ID NOs: 31, 32 and 30.

Preferably, the humanized monoclonal antibody (mAb) of the present invention has the heavy chain region comprising at least two of the three CDRs shown by SEQ ID NOs: 7, 8 and 11, and/or the light chain region comprising at least two of the three CDRs shown by SEQ ID NOs: 33, 34 and 30.

Preferably, the humanized monoclonal antibody (mAb) of the present invention has the heavy chain region comprising at least two of the three CDRs shown by SEQ ID NOs: 7, 8 and 12, and/or the light chain region comprising at least two of the three CDRs shown by SEQ ID NOs: 35, 34 and 30.

Preferably, the humanized monoclonal antibody (mAb) of the present invention has the heavy chain region comprising at least two of the three CDRs shown by SEQ ID NOs: 1, 13 and 136, and/or the light chain region comprising at least two of the three CDRs shown by SEQ ID NOs: 39, 40 and 38.

Preferably, the humanized monoclonal antibody (mAb) of the present invention has the heavy chain region comprising at least two of the three CDRs shown by SEQ ID NOs: 1, 13 and 136, and/or the light chain region comprising at least two of the three CDRs shown by SEQ ID NOs: 41, 42 and 38.

Preferably, the humanized monoclonal antibody (mAb) of the present invention has the heavy chain region comprising at least two of the three CDRs shown by SEQ ID NOs: 1, 13 and 136, and/or the light chain region comprising at least two of the three CDRs shown by SEQ ID NOs: 43, 44 and 38.

Preferably, the humanized monoclonal antibody (mAb) of the present invention has the heavy chain region comprising at least two of the three CDRs shown by SEQ ID NOs: 1, 15 and 16, and/or the light chain region comprising at least two of the three CDRs shown by SEQ ID NOs: 39, 40 and 38.

Preferably, the humanized monoclonal antibody (mAb) of the present invention has the heavy chain region comprising at least two of the three CDRs shown by SEQ ID NOs: 1, 15 and 16, and/or the light chain region comprising at least two of the three CDRs shown by SEQ ID NOs: 45, 42 and 46.

Preferably, the humanized monoclonal antibody (mAb) of the present invention has the heavy chain region comprising at least two of the three CDRs shown by SEQ ID NOs: 1, 15 and 16, and/or the light chain region comprising at least two of the three CDRs shown by SEQ ID NOs: 47, 44 and 46.

Preferably, the humanized monoclonal antibody (mAb) of the present invention has the heavy chain region comprising at least two of the three CDRs shown by SEQ ID NOs: 17, 18 and 20, and/or the light chain region comprising at least two of the three CDRs shown by SEQ ID NOs: 50, 40 and 51.

Preferably, the humanized monoclonal antibody (mAb) of the present invention has the heavy chain region comprising at least two of the three CDRs shown by SEQ ID NOs: 17, 18 and 20, and/or the light chain region comprising at least two of the three CDRs shown by SEQ ID NOs: 50, 40 and 52.

Preferably, the humanized monoclonal antibody (mAb) of the present invention has the heavy chain region comprising at least two of the three CDRs shown by SEQ ID NOs: 17, 18 and 20, and/or the light chain region comprising at least two of the three CDRs shown by SEQ ID NOs: 50, 40 and 53.

Preferably, the humanized monoclonal antibody (mAb) of the present invention has the heavy chain region comprising at least two of the three CDRs shown by SEQ ID NOs: 17, 18 and 20, and/or the light chain region comprising at least two of the three CDRs shown by SEQ ID NOs: 54, 42 and 55.

Preferably, the humanized monoclonal antibody (mAb) of the present invention has the heavy chain region comprising at least two of the three CDRs shown by SEQ ID NOs: 17, 18 and 20, and/or the light chain region comprising at least two of the three CDRs shown by SEQ ID NOs: 56, 44 and 55.

Preferably, the humanized monoclonal antibody (mAb) of the present invention comprises constant region derived from human constant region.

Preferably, the humanized monoclonal antibody (mAb) of the present invention has the human light chain constant region derived from kappa light chain constant region.

Preferably, the humanized monoclonal antibody (mAb) of the present invention has the human heavy chain constant region derived from a human IgG1, IgG2, IgG3, or IgG4 heavy chain constant region.

In some embodiments, the anti-AREG antibody or fragment thereof according to the present invention is capable of blocking binding of AREG to EGFR.

In some embodiments, the anti-AREG antibody or fragment thereof according to the present invention is capable of inhibiting EGFR phosphorylation.

In another aspect, the present invention provides an isolated polynucleotide or a nucleic acid encoding the anti-AREG antibody or fragment thereof according to the present invention.

In some embodiments, the polynucleotide according to the present invention may encode the entire heavy chain variable region, or the entire light chain variable region, or the both on the same polynucleotide molecule or on separate polynucleotide molecules.

Alternatively, the polynucleotide according to the present invention may encode portions of heavy chain variable region, or the light chain variable region, or the both on the same polynucleotide molecule or on separate polynucleotide molecules.

In some embodiments, the polynucleotide according to the present invention comprises the DNA sequence encoding the heavy chain variable region shown by any one of sequences SEQ ID NOs: 90, 92, 94, 96, 98, 100, 102, 104, 106, 108, 112, 115, and 117, and/or the DNA sequence encoding the light chain variable region shown by any one of sequences SEQ ID NOs: 91, 93, 95, 97, 99, 101, 103, 105, 107, 109, 110, 111, 113, 114, 116, 118, 119, 120, 121, and 122.

In another aspect, the present invention provides an isolated cell, or vector comprising one or more polynucleotide encoding the anti-AREG antibody or fragment thereof according to the present invention.

In some embodiments, the cell is a hybridoma cell producing the anti-AREG antibody or fragment thereof according to the present invention.

In another aspect, the present invention provides a composition comprising the anti-AREG antibody or fragment thereof according to the present invention and a pharmaceutical acceptable carrier.

In another aspect, the present invention provides use of the anti-AREG antibody or fragment thereof according to the present invention in manufacturing a medicament for treating a disorder in a subject, whose AREG is overexpressed, upregulated or activated.

The subject may be a mammalian subject, for whom, diagnosis, prognosis, or therapy is desired. Mammalian subjects include humans, domestic animals, farm animals, and zoo, sport, or pet animals, such as dogs, cats, guinea pigs, rabbits, rats, mice, horses, cattle, cows, and so on.

The disorder is a fibrotic disease including but not limited to renal fibrosis, hepatic fibrosis, pulmonary fibrosis, in particular, IPF.

In another aspect, the present invention provides a method for treating a disorder in a subject, whose AREG is overexpressed, upregulated or activated, comprising administering to the patient the anti-AREG antibody or fragment thereof according to the present invention. The disorder is a fibrotic disease including but not limited to renal fibrosis, hepatic fibrosis, pulmonary fibrosis, in particular, IPF.

The subject may be a mammalian subject, for whom, diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

In another aspect, the present invention provides a method for determining the presence of AREG protein, comprising exposing a cell suspected of containing AREG protein to the anti-AREG antibody or fragment thereof according to the present invention, and determining binding of the anti-AREG antibody or fragment thereof to the cell.

The method may be a method for diagnosing a disorder in a subject, whose AREG is overexpressed, upregulated or activated. The disorder is a fibrotic disease including but not limited to renal fibrosis, hepatic fibrosis, pulmonary fibrosis, in particular, IPF.

The subject may be a mammalian subject, for whom, diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

In another aspect, the present invention provides an isolated AREG protein, having an amino acid sequence shown in any one of SEQ ID NOs: 123-132, or an amino acid sequence that is at least 85% identical to any one of SEQ ID NOs: 123-132.

The isolated AREG protein can be used as epitope for producing the anti-AREG antibody or fragment thereof according to the present invention.

The isolated AREG protein according to the present invention can be used to identify anti-AREG antibody or fragment thereof having no or weak cross-reactivity to murine AREG.

Two amino acids (E149 and H164, based on hAREG numbering) are identified as critical epitope residues for binding the anti-AREG antibody or fragment thereof according to the present invention to hAREG rather than mAREG. The amino acids, K149 and N164 in mAREG (based on hAREG numbering), are residues responsible for the anti-AREG antibody or fragment thereof according to the present invention lack of cross reactivity to mAREG, and the 164N residue is the most critical one.

Preferably, isolated AREG protein has the amino acid Glu149 (using hAREG numbering), and/or His164 (using hAREG numbering).

In another aspect, the present invention provides use of the isolated AREG protein according to the present invention for identifying anti-AREG antibody or fragment thereof binding to hAREG, and having no or weak cross-reactivity to mAREG.

### Definitions

The terms “AREG” and “Areg” as used herein refer to “Amphiregulin” or the gene encoding Amphiregulin, and are used interchangeably. “AREG (Areg)” is a member of the epidermal growth factor (EGF) family, and a low affinity ligand for EGF Receptor (EGFR). Unless otherwise stated in the Description, “AREG (Areg)” indicates human AREG (Areg). The binding of EGFR to AREG activates major intracellular signaling cascades governing cell survival, proliferation and motility. AREG protein is synthesized from a 252 amino acid transmembrane precursor (pro-AREG) (SEQ ID NO: 135), which is subjected to proteolytic cleavage within its ectodomain by cell membrane proteases, mainly TACE/ADAM17, thereby releasing two soluble forms of AREG protein, wherein the larger one corresponds to residues 101-184 of pro-AREG (SVRVEQVVKPPQNKTESENTSDKPKRKKKGGKNGKNRRNRKK KNPCNAEFQNFCIHGECKYIEHLEAVTCKCQQEYFGERCGEK), and the shorter one corresponds to residues 107-184 of pro-AREG (78 residues in length). AREG protein contains a heparin binding domain (corresponding to residues 101-143 of pro-AREG, SVRVEQVVKPPQNKTESENTSDKPKRKKKGGKNGKNRRNRK) and an EGF-like domain (corresponding to residues 144-184 of pro-AREG, KKNPCNAEFQNFCIHGECKYIEH LEAVTCKCQQEYFGERCGEK). Pro-AREG activates EGFR on adjacent cells in a juxtacrine mode; while the soluble forms of AREG activate EGFR in an autocrine or a paracrine mode.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term “isolated” as used herein with respect to cells, polynucleotides, such as DNA or RNA, proteins or polypeptides, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. Isolated polynucleotides refer to molecules separated from other DNAs or RNAs, respectively, and are present in the natural source of the macromolecule. Isolated polypeptides are meant to encompass both purified and recombinant polypeptides.

The products and methods disclosed herein encompass polypeptides and polynucleotides having the sequences specified, or sequences identical or similar thereto, e.g., sequences having at least about 85% or 95% sequence identity (identical) to the sequence specified. In the context of an amino acid sequence, the term “85% or 95% sequence identity(identical)” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 86%, 87%, 88%, 89%, 90%, 91%, 92%, 93%, 94%, 95%, 96%, 97%, 98% or 99% identity to a reference sequence, e.g., a sequence provided herein.

In the context of nucleotide sequence, the term “85% or 95% sequence identity (identical)” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 86%, 87%, 88%, 89%, 90%, 91%, 92%, 93%, 94%, 95%, 96%, 97%, 98% or 99% identity to a reference sequence, e.g., a sequence provided herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The terms “polypeptide”, “peptide” and “protein” are used interchangeably herein to refer to polymers of amino acids of any length.

The terms “nucleic acid”, “nucleic acid sequence”, “nucleotide sequence”, or “polynucleotide sequence,” and “polynucleotide” are used interchangeably.

As used herein, the term “antibody or antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain. As used herein, an antibody molecule “binds to” an antigen as such binding is understood by one skilled in the art. In one embodiment, an antibody binds to an antigen with a dissociation constant (KD) of about 1×10⁻⁵M or less, 1×10⁻⁶M or less, or 1×10⁻⁷M or less, 1×10⁻⁸M or less, 1×10⁻⁹M or less, 1×10⁻¹⁰M or less, 1×10⁻¹¹M or less.

For example, an antibody molecule can include a heavy (H) chain variable domain sequence (abbreviated herein as VH), and a light (L) chain variable domain sequence (abbreviated herein as VL). In an embodiment, an antibody molecule comprises or consists of a heavy chain and a light chain. In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

The terms “antibody fragment” or “antigen-binding fragment”, as used herein, is a portion of an antibody such as F(ab′)₂, F(ab)₂, Fab′, Fab, Fv, scFv and the like. An antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Examples of antigen-binding fragments of an antibody molecule include: (i) a Fab fragment, a monovalent fragment consisting of the VL, VH, CK and CH domains; (ii) a F(ab′)₂ fragment, a bivalent fragment comprising two Fab fragments linked by a disulfide bridge at the hinge region; (iii) a Fd fragment consisting of the VH and CH domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv(scFv); (viii) a single domain antibody. These antibody fragments may be obtained using any suitable method, including conventional techniques known to those with skill in the art, and the fragments can be screened for utility in the same manner as are intact antibodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art.

The light and heavy chains are divided into regions of “constant” and “variable”. The variable domains of both the light (VL) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. The N-terminal portion is a variable region and at the C-terminal portion is a constant region; the CH3 and CK domains actually comprise the carboxy-terminus of the heavy and light chain, respectively.

The variable region allows the antibody to selectively recognize and specifically bind epitopes on antigens. The VL domain and VH domain, or subset of the complementarity determining regions (CDRs), of an antibody combine to form the variable region that defines a three dimensional antigen-binding site. This quaternary antibody structure forms the antigen-binding site present at the end of each arm of Y. More specifically, the antigen-binding site is defined by three CDRs on each of the VH and VK chains (i.e., HCDR1, HCDR2, HCDR3, LCDR1, LCDR2 and LCDR3).

The terms “complementarity determining region” and “CDR” as used herein refer to the sequences of amino acids within antibody variable regions which confer antigen specificity and binding affinity. In some embodiments, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of well-known schemes, including those described by Kabat et al. (1991), “Sequences of Proteins of Immunological Interest,” 5th Ed. Public Health Service, National Institutes of Health, Bethesda, Md. (“Kabat” numbering scheme).

Each VH and VL typically includes three CDRs and four FRs, arranged from amino-terminus to carboxy-terminus in the following order: FR1, CDR1, FR2, CDR2, FR3, CDR3, FR4.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

As used herein, the term “epitope” refers to the moieties of an antigen (e.g., human AREG (hAREG)) that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or mutagenesis. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., library selection, and screening, or recombinant methods).

The antibody molecule can be a polyclonal or a monoclonal antibody. In other embodiments, the antibody can be recombinantly produced, e.g., produced by yeast display, phage display, or by combinatorial methods.

In one embodiment, the antibody is a fully human antibody (e.g., an antibody produced by yeast display, an antibody produced by phage display, or an antibody made in a mouse which has been genetically engineered to produce an antibody from a human immunoglobulin sequence), or a non-human antibody, e.g., a murine (mouse or rat), goat, primate (e.g., monkey), or camel antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein.

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to AREG. In some embodiments, the donor is a murine antibody, e.g., a rat or mouse antibody, and the recipient is a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., murine). The acceptor framework is a naturally-occurring (e.g., a human) framework or a consensus framework, or a sequence about 85% or higher, e.g., 90%, 95%, 99% or higher identical thereto.

An antibody can be humanized by methods known in the art. Humanized or CDR-grafted antibodies can be produced by CDR-grafting or CDR substitution, wherein one, two, or all CDRs of an immunoglobulin chain can be replaced.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (Eu numbering) in human IgG4, are also contemplated.

It is understood that the molecules of the invention may have additional conservative or nonessential amino acid substitutions, which do not have a substantial effect on their functions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Conservative amino acid substitutions:

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

### BRIEF DESCRIPTION OF THE DRAWINGS

FIG. 1 shows the binding of E1H3L4 and P7 to hAREG, mAREG and hAREG-C18.

FIG. 2 shows inhibition activities of anti-AREG mAbs against EGFR phosphorylation in hEGFR-expressing epidermoid carcinoma cells.

FIG. 3 shows five hAREG-EGFd variants generated by changing each amino acid at five different sites of hAREG-EGFd to the counterpart amino acid of mAREG-EGFd.

FIG. 4 shows the scheme of generating a mouse line in which Cdc42 gene is specifically deleted in AT2 cells. The mice in which the exon2 of the Cdc42 gene is specifically deleted in AT2 cells are named as Cdc42 AT2 null mice.

FIG. 5 shows that loss of Cdc42 in AT2 cells leads to progressive lung fibrosis in PNX-treated mice.

FIG. 6 shows that the anti-AREG antibody (P7) is effective for treating lung fibrosis in the IPF-like lung fibrosis mouse model.

FIG. 7 shows that the anti-AREG antibody (E1H3L4) treatment could accelerate the recovery of mice in the bleomycin-induced lung fibrosis mouse model.

FIG. 8 shows that the anti-AREG antibody (E1H3L4) is effective for treating lung fibrosis in the IPF-like lung fibrosis mouse model.

FIG. 9 shows that the anti-AREG antibody hu9C12v4 significantly prolongs the life expectancy of fibrosis mice in the IPF-like lung fibrosis mouse model.

### DESCRIPTION OF PARTICULAR EMBODIMENTS OF THE INVENTION

The descriptions of particular embodiments and examples are provided by way of illustration and not by way of limitation. Those skilled in the art will readily recognize a variety of noncritical parameters that could be changed or modified to yield essentially similar results.

### EXAMPLES

### Example 1. Generation of Human mAbs Against AREG from a Phage Library

1. Preparation of Soluble AREG Proteins or Peptides for Library Selection, and Screening

The DNA sequences encoding three forms of AREG proteins (listed below) were cloned into a prokaryotic expression vector(pETDuet), and expressed as a fusion protein carrying N-terminal tags (His₆, thioredoxin(TRX), a HRV 3C Protease cleavage site, and an Avi tag). The proteins were expressed in Escherichia coli(TransB) by IPTG induction, and purified from the supernatant of cell lysate using Ni-NTA beads, cleaved by HRV 3C Protease, then was biotinylated using BirA enzyme.

The three AREG proteins are:
hAREG: human AREG comprising residues 101-184 of the human pro-AREG with an N-terminal AVI tag (GLNDIFEAQKIEWHE). The amino acid sequence amino acid sequence of hAREG residues 101-184 is:

(SEQ ID NO: 129)
SVRVEQVVKPPQNKTESENTSDKPKRKKKGGKNGKNRRNRKKKNPCNA
EFQNFCIHGECKYIEHLEAVTCKCQQEYFGERCGEK

mAREG: mouse AREG comprising residues 94-177 of the mouse pro-AREG with an N-terminal AVI tag. The amino acid sequence is:

(SEQ ID NO: 130)
GLNDIFEAQKIEWHEGGGGSGGSVRVEQVIKPKKNKTEGEKSTEKPKR
KKKGGKNGKGRRNKKKKNPCTAKFQNFCIHGECRYIENLEVVTCNCHQ
DYFGERCGEK

mAREG-EGFd: EGF-like domain of mouse AREG comprises residues 135-177 of the mouse Pro-AREG with an N-terminal AVI tag. The amino acid sequence of mAreg residues 135-177 is:

(SEQ ID NO: 131)
KKNPCTAKFQNFCIHGECRYIENLEVVTCNCHQDYFGERCGEK.

In addition, a biotinylated peptide, C18 was synthesized(Scilight-peptide, Beijing, China). C18 comprises 14 amino acids of the C-terminus of human AREG (residues 171-184) and a linker (residues GSSG) at the N-terminus. The sequence of C18 is GSSGKCQQEYFG ERCGEK (SEQ ID NO:132).

2. Selection and Further Characterization of Antibodies from Phage Display Antibody Library

Phage Display Antibody Library

A human non-immune scFv(Single-chain variable fragment) antibody library was constructed from peripheral blood mononuclear cells (PBMCs) of 93 healthy donors. The library has a size of a total of 1.1×10¹⁰ members (Li et al., 2017).

Selection and Screening of Phage Antibody Library

Phage particles expressing scFv on their surface (phage-ScFv) were prepared from the library and used for selection of scFvs against the target antigens including the biotinylated AREG proteins and peptides. The antigens were captured on streptavidin-conjugated magnetic M-280 Dynabeads® (Life Technologies) and then incubated with 5×10¹² phage particles prepared from the library, respectively. For each soluble AREG protein or peptide (antigen, Ab), two rounds of selection were performed. For obtaining cross-reactive human mAbs recognizing both hAREG and mAREG, the hAREG and mAREG were used respectively in the 1^{st} and 2^{nd} rounds of selection. After the 2^{nd} round of selection, about 400 phage-Ab clones were screened for cross-binding activity to both hAREG and mAREG using ELISA, and clones with cross-binding activity or high binding affinity to hAREG were selected for sequencing analysis to identify clones with different antibody sequences, including variable regions of heavy (VH) and light (VL) chain. Some of the phage-Abs were subsequently converted into human IgG1 (hIgG1) or mouse IgG2a format and analyzed for binding to both hAREG and mAREG using enzyme-linked immunosorbent assay (ELISA) or Biacore.

3. Preparation of Full-Length Antibody

The VH and VL coding sequence of a scFv was separately sub-cloned into antibody heavy chain (HC) expression vector(plasmid) and light chain (LC) expression vector(plasmid). To make full-length antibodies, 293F cells were transiently co-transfected with the two expression plasmids (HC+LC plasmids) at a 1:1 ratio. Six days after transfection, the cell culture supernatant was harvested for purification of antibodies by Protein A affinity chromatography.

4. ELISA Assay

Streptavidin (Sigma, 4 g/mL) in phosphate buffered saline (PBS) was coated in U-bottom 96-well plate (Nunc, MaxiSorp™), 100 μL per well, at 4° C. overnight or 37° C. for 1 hour.

About 0.5 g/mL of AREG protein or peptide at 100 μL per well were then captured onto the plates by incubation at 30° C. for 1 hour. For phage-scFv based ELISA, serial diluted phage-scFvs in PBS containing 2% nonfat milk were added to each well at 100 μL per well. Specific bound phage-scFvs were detected by adding HRP-conjugated mouse anti-M13 antibody (GE Healthcare) and incubated for 30 mins at 30° C. In between each incubation step, the ELISA plate was washed for 6 times with PBST solution (0.05% Tween20 containing PBS) at 300 L per well. Followed by HRP-conjugated antibody incubation, the ELISA signal was developed by incubating with TMB substrate (Sigma) for 5-10 mins at 30° C. and then the reaction was stopped with 2M H₂SO₄ at 50 L per well. The absorbance at 450 nm with the correction wavelength set at 630 nm was read by a microplate reader (Bio-Rad). For IgG based ELISA, the method was basically the same as described above for phage-scFvs except the bound antibodies were detected by HRP-conjugated mouse anti-Fc secondary antibody (Thermo Fisher Scientific).

5. E1L2 Antibody Engineering for Improved Affinity and Solubility

To improve the affinity of E1L2 antibody, the VH-CDR3 and VL-CDR3 of E1L2 were engineered separately. For VH-CDR3, a phage display sub-library with random mutagenesis for the HCDR3 of E1L2 was constructed. Antibody sub-library selection and screening were done similarly as described above for screening of antibody library against AREG. To obtain high-affinity hits, competitive elution with E1L2 full-length mAb was used. Subsequently, single clones were picked and rescued to produce phage-scFvs in the bacterial culture supernatant to screen for binding to hAREG. Only hits with higher binding affinities than E1L2 were retained. For VL-CDR3, it was engineered with specific amino acid mutations based on structural modeling. To improve solubility, the engineered VL CDRs of E1L2 were grafted to human IGLV1-44*01 germline.

6. SPR Measurement of Affinity of Human mAbs

To evaluate affinities of human mAbs, SPR measurement was performed using Biacore T200 instrument. mAbs were captured on anti-human Fc CM5 biosensor chip surface, the EGF domain of hAREG (aa142-184) or mAREG (aa135-177) fused with mFc tag (hAREG-EGFd-mFc or mAREG-EGFd-mFc) were examined for binding to the mAbs. The fusion proteins in serial dilutions were injected over antibody-bound surface, followed by a dissociation phase. Association rates (Ka) and dissociation rates (Kd) were calculated using the one-to-one Langmuir binding model (BIA Evaluation Software, GE Life Sciences). The equilibrium dissociation constant (KD) was calculated as the ratio kd/ka.

7. Results

Generation of Human mAbs, E1L2 and P7, Against AREG from a Phage Library

By using the above described phage antibody library selection and ELSIA screening, we identified C1, E1L2, P5, P6, P7 and P10 anti-AREG human mAbs. Specifically, by using E. coli expressed biotinylated hAREG and mAREG proteins in the 1^{st} and 2^{nd} round of the library selection, respectively, we identified C1 antibody. By using the biotinylated hAREG and biotinylated mAREG-EGFd (expressed in E. coli) in the 1^{st} and 2^{nd} round, respectively, we identified E1L2 antibody. By using hAREG derived C18 peptide as the target for library selection for two rounds, we identified P5, P6, P7, and P10; E1L2 was also screened out from this library selection. Among these antibodies, E1L2 and P7 antibodies were selected for further characterization based on their binding specificity and affinity to both hAREG and mAREG.

Creation of E1L2-Derived Antibody E1H3L4 with Improved Affinity and Solubility

The binding affinity of E1L2 was further improved by VH-CDR3 and VL-CDR3 engineering. The solubility of the engineered E1L2 was improved by grafting its VL-CDRs to human IGLV1-44*01 germline, thus, resulting in an antibody, E1H3L4. Comparing to E1L2, E1H3L4 has three amino-acid changes in the VH-CDR3 and four amino-acid changes in the VL-CDR3. Specifically, amino acids at positions 100-100, (Kabat system)correct in VH-CDR3 of E1H3L4 are SYNN, while they are GYDY in E1L2 antibody; amino acids at positions 93-95a (Kabat system) in VL-CDR3 of E1H3L4 are KNNK, while they are SGLN in E1L2 antibody. The CDRs of E1L2, E1H3L4 and P7 were listed in Table 1. The nucleotide sequences and the amino acid sequences of VH and VL of E1L2, E1H3L4 and P7 were listed in Table 2.

TABLE 1
CDRs of E1L2, E1H3L4, and P7

| | HCDR1 | HCDR2 | HCDR3 |
|---|---|---|---|
| | (SEQ ID No.) | (SEQ ID No.) | (SEQ ID No.) |
| E1L2 | SYAMS(1) | AISGSGGSTYYA | PTSRYSYGYDY |
| | | DSVKG(2) | (3) |
| E1H3L4 | SYAMS(1) | AISGSGGSTYYA | PTSRYSYSYNN |
| | | DSVKG(2) | (4) |
| P7 | SHAMS(5) | AISGSGGSTYYA | VDTKFDP(6) |
| | | DSVKG(2) | |
| | LCDR1 | LCDR2 | LCDR3 |
| | (SEQ ID No.) | (SEQ ID No.) | (SEQ ID No.) |
| E1L2 | TGNSNNVGDQGA | RNNNRPS(22) | STWDSGLNSVV |
| | V(21) | | (23) |
| E1H3L4 | TGNSNNVGDQGA | RNNNRPS(22) | STWDKNNKSVV |
| | V(21) | | (24) |
| P7 | SGSSSNIGSNTV | SNNQRPS(26) | EVWDDSLNGPV |
| | N(25) | | (27) |

The differences between E1L2 and E1H3L4 were underlined.
CDRs are defined using Kabat system.

TABLE 2

| Name | VH | VH | VL | VL |
|---|---|---|---|---|
| of the | (nucleotide | (amino acid | (nucleotide | (amino acid |
| Antibody | sequence) | sequence) | sequence) | sequence) |
| E1L2 | SEQ ID | SEQ ID | SEQ ID | SEQ ID |
| | NO: 90 | NO: 57 | NO: 91 | NO: 70 |
| E1H3L4 | SEQ ID | SEQ ID | SEQ ID | SEQ ID |
| | NO: 92 | NO: 58 | NO: 93 | NO: 71 |
| P7 | SEQ ID | SEQ ID | SEQ ID | SEQ ID |
| | NO: 94 | NO: 59 | NO: 95 | NO: 72 |

8. Further Characterization of E1H3L4 and P7 mAbs

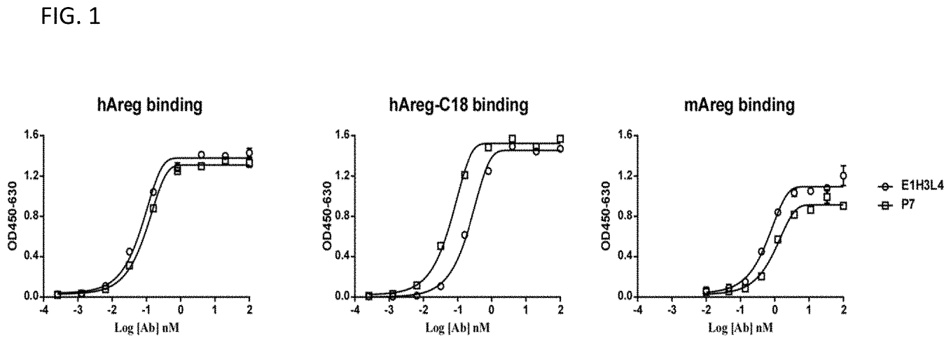

Comparing the binding of E1H3L4 and P7 to mAREG, E1H3L4 showed slightly stronger binding to mAREG than P7. Both of the two antibodies bound to the C-terminal peptide (C18, aa 171-184) within the EGF domain as expected since the C18 peptide was the target used in the library selection (FIG. 1).

### Example 2. Generation of mAbs Against AREG Using Mouse Hybridoma Method and Humanization of the Mouse mAbs

1. Preparation of Antigens for Immunization of Mice or SPR Analysis

Human AREG (hAREG) EGF-like domain fused with an Fc fragment of human IgG1 or mouse IgG2a was expressed in 293F as a fusion protein, named as hAREG-EGFd-hFc and hAREG-EGFd-mFc, respectively. 72 hours after transfection, the cell culture supernatant were harvested for purification of the Fc-fusion AREG proteins by Protein A affinity chromatography.

2. Generation of Anti-hAREG EGF Domain Monoclonal Antibodies

Six week-old Balb/c mice (from Beijing Vital River Laboratory Animal Technology Co., Ltd.) were immunized by subcutaneously administration with 100 μl of 1:1 antigen/adjuvant emulsion containing 50 μg of hAREG-EGFd-mFc. For priming immunization, complete Freund's adjuvant (Sigma) was used. For boosting immunization, incomplete Freund's adjuvant (Sigma) was used. Boosting immunization was performed every two weeks. After the 3^{rd} boosting immunization, the sera of the mice were evaluated for binding to biotinylated hAREG by ELISA one week after each immunization. Mice with high titers of anti-hAREG antibody were boosted intraperitoneally with 50 μg of hAREG-EGFd-mFc without adjuvant. Three days after boosting, spleenocytes were isolated and fused with SP2/0 cells, following standard hybridoma fusion methods.

The supernatant of hybridoma clones were examined for binding activity to biotinylated hAREG by ELISA. Clones with high binding activity were selected and expanded for subsequent subcloning, and the supernatant of the subclones were analyzed by ELISA and SPR. The SPR analysis was performed using Biacore T200 instrument (GE Life Sciences). Diluted supernatant was captured on anti-mFc CM5 biosensor chip, then 200 nM of hAREG EGF domain-hFc flowed in mobile phase. Subclones with high affinity was expanded for RNA extraction. Cells were resuspended in TRIzol (Life Technologies), and total RNA was extracted following the instruction manual. The cDNA of subclones was synthesized using PrimeScript™ RT Master Mix (TaKaRa). The VH and VL genes of each antibody were amplified using a set of PCR primers specific to mouse antibody variable genes. PCR products were cloned into a PCR sequencing vector for sequencing.

3. Humanization of Anti-hAREG EGF-Like Monoclonal Antibodies

For humanization of the AREG mAbs, sequences of murine mAbs were searched for human germline IgG genes homologous to identify the human germline genes with high homology to the murine mAbs (9C12, 23H8 and 1H9), and then chosen them as templates for humanization. Humanization was carried out by complementarity-determining region (CDR)-grating, specifically by grafting CDRs of murine mAb onto human acceptor framework of the selected human germline gene templates. This humanization process was also guided by the simulated 3D structure of each antibody and human framework residues back mutation to murine residues in order to maintain the overall antibody and CDR loop structures as well as AREG binding affinity.

4. hu9C12v1 Antibody Sub-Library Construction and Selection for Improving Affinity

To improve the affinity of antibody, two phage display sub-libraries with random mutagenesis for the HCDR3 and LCDR1 of hu9C12v1 were constructed through NNK degenerate codons. Antibody sub-library selection and screening were done similarly as described above for screening of antibody library against AREG and affinity improvement of E1L2 antibody. Only hits with higher binding affinities than hu9C12v1 were retained after the screening.

5. SPR Measurement Affinity of the mAbs

To evaluate affinities of different mouse hybridoma mAbs or their humanized and engineered variants, SPR measurement was performed using Biacore T200 instrument. mAbs were captured on anti-human Fc CM5 biosensor chip surface, hAREG-EGFd-mFc, mAREG-EGFd-mFc or hAREG-98aa (purchased from PeproTech, cat #100-55B) in serial dilutions were injected over antibody-bound surface, followed by a dissociation phase. Association rates (Ka) and dissociation rates (Kd) were calculated using the one-to-one Langmuir binding model (BIA Evaluation Software, GE Life Sciences). The equilibrium dissociation constant (KD) was calculated as the ratio kd/ka.

6. Results

Generation of Anti-hAREG EGF Domain Monoclonal Antibodies

Anti-hAREG mAbs were generated based on conventional hybridoma fusion technology. MAbs with high binding activities in ELISA and SPR assay were selected for further characterization. Through screening thousands of hybridoma clones, we identified a panel of mAbs with high binding affinity to hAREG. Three top mAbs, 9C12, 23H8 and 1H9, were selected for further analysis based on their unique sequences, binding affinity and high yield of recombinant antibody production. These antibodies were made as mouse antibodies of mIgG1 or mIgG2a isotype or chimeric antibodies (mouse variable region grafted onto human IgG1 constant regions) by recombinant expression.

Humanization of 9C12 or Creation of Humanized Antibody Variants with Improved Binding Affinity to hAREG or mAREG and Improved Physicochemical Properties

CDR-grafting and structural modeling were used to generate the first version of the humanized 9C12, hu9C12v1, which has comparable affinity to hAREG as the chimeric antibody, ch9C12 (having the variable regions of 9C12, and constant regions of human IgG1). To improve the affinity of hu9C12v1 for hAREG, two phage display sub-libraries with randomized mutations within its HCDR3 and LCDR3 regions were separately constructed. After stringent bio-panning selections, a small panel of affinity-improved antibodies was obtained. Based on the sequences of this panel of antibodies, three mAbs, hu9C12v4, hu9C12v5 and hu9C12v6, were created to improve the binding affinity to hAREG or mAREG, and to improve their physicochemical properties. Comparing to hu9C12v1, hu9C12v4 has one amino-acid difference in the VH-CDR2, six amino-acid differences in the VK-CDR1, and two amino-acid differences in the VK-CDR2; hu9C12v5 has five amino-acid differences in the VH-CDR3, five amino-acid differences in the VK-CDR1, and one amino-acid difference in the VK-CDR2; hu9C12v6 has two amino-acid differences in the VH-CDR3, five amino-acid differences in the VK-CDR1, and one amino-acid difference in the VK-CDR2. The CDRs of the three mAbs were compared to the murine antibody as shown in Table 3. The nucleotide sequences and the amino acid sequences of VH and VL of the three mAbs and murine antibody were listed in Table 4. The SPR-determined binding affinities of the three mAbs to hAREG or mAREG were listed in Tables 5-6.

TABLE 3
Comparison of CDRs among
different versions of mAb 9C12

| | HCDR1 | HCDR2 | HCDR3 |
|---|---|---|---|
| | (SEQ ID No.) | (SEQ ID No.) | (SEQ ID No.) |
| m9C12 | SYPMS(7) | TISTGGTYTYYP | QGPIYYGNYYYA |
| | | DSVKG(8) | MDY(9) |
| 9C12v1 | SYPMS(7) | TISTGGTYTYYP | QGPIYYGNYYYA |
| | | DSVKG(8) | MDY(9) |
| hu9C12v4 | SYPMS(7) | TISTGGRYTYYP | QGPIYYGNYYYA |
| | | DSVKG(10) | MDY(9) |
| hu9C12v5 | SYPMS(7) | TISTGGTYTYYP | QGPILRKNYYYG |
| | | DSVKG(8) | MDV(11) |
| hu9C12v6 | SYPMS(7) | TISTGGTYTYYP | QGPIYYGNYYYG |
| | | DSVKG(8) | MDV(12) |
| | LCDR1 | LCDR2 | LCDR3 |
| | (SEQ ID No.) | (SEQ ID No.) | (SEQ ID No.) |
| m9C12 | RSSQSLVHSDGN | KVSNRFS(29) | SQSTHVPYT |
| | TYLH(28) | | (30) |
| hu9C12v1 | RSSQSLVHSDGN | KVSNRFS(29) | SQSTHVPYT |
| | TYLH(28) | | (30) |
| hu9C12v4 | RSSQSLVDGEDG | KVSERFD(32) | SQSTHVPYT |
| | TYLN(31) | | (30) |
| hu9C12v5 | RSSQSLVDGQDG | KVSNRFD(34) | SQSTHVPYT |
| | TYLH(33) | | (30) |
| hu9C12v6 | RSSQSLVNQEGE | KVSNRFD(34) | SQSTHVPYT |
| | TYLH(35) | | (30) |

The differences between Abs were underlined.
CDRs are defined using Kabat system.

TABLE 4

| Name | VH | VH | VL | VL |
|---|---|---|---|---|
| of the | (nucleotide | (amino acid | (nucleotide | (amino acid |
| Antibody | sequence) | sequence) | sequence) | sequence) |
| m9C12 | SEQ ID | SEQ ID | SEQ ID | SEQ ID |
| | NO: 96 | NO: 60 | NO: 97 | NO: 73 |
| hu9C12v1 | SEQ ID | SEQ ID | SEQ ID | SEQ ID |
| | NO: 98 | NO: 61 | NO: 99 | NO: 74 |
| hu9C12v4 | SEQ ID | SEQ ID | SEQ ID | SEQ ID |
| | NO: 100 | NO: 62 | NO: 101 | NO: 75 |
| hu9C12v5 | SEQ ID | SEQ ID | SEQ ID | SEQ ID |
| | NO: 102 | NO: 63 | NO: 103 | NO: 76 |
| hu9C12v6 | SEQ ID | SEQ ID | SEQ ID | SEQ ID |
| | NO: 104 | NO: 64 | NO: 105 | NO: 77 |

TABLE 5

| | hAREG-EGFd | | | mAREG-EGFd | | |
|---|---|---|---|---|---|---|
| mAbs | Kₐ (M⁻¹, s⁻¹) | K_{off} (s) | K_{D} (M) | Kₐ (M⁻¹, s⁻¹) | K_{off} (s) | K_{D} (M) |
| ch9C12 | 5.87 × 10⁵ | 1.0 × 10⁻³ | 1.71 × 10⁻⁹ | | | |
| hu9C12v1 | 5.70 × 10⁵ | 5.72 × 10⁻⁴ | 1.00 × 10⁻⁹ | | | |
| hu9C12v4 | 7.45 × 10⁵ | 7.70 × 10⁻⁴ | 1.04 × 10⁻⁹ | 2.96 × 10⁵ | 2.67 × 10⁻³ | 9.01 × 10⁻⁸ |
| hu9C12v5 | 4.83 × 10⁵ | 4.11 × 10⁻⁴ | 8.52 × 10⁻¹⁰ | | | |
| hu9C12v6 | 9.78 × 10⁵ | 3.89 × 10⁻⁴ | 3.98 × 10⁻¹⁰ | | | |

TABLE 6

| | hAREG-98aa | | |
|---|---|---|---|
| mAbs | Kₐ (M⁻¹, s⁻¹) | K_{off} (s) | K_{D} (M) |
| hu9C12v4 | 8.04 × 10⁶ | 3.72 × 10⁻³ | 4.62 × 10⁻¹⁰ |
| hu9C12v5 | 8.11 × 10⁶ | 2.83 × 10⁻³ | 3.49 × 10⁻¹⁰ |
| hu9C12v6 | 2.43 × 10⁷ | 4.57 × 10⁻³ | 1.88 × 10⁻¹⁰ |

Humanization of 23H8

We used CDR-grafting and structural modeling to generate the humanized 23H8 mAbs. The human VH germline gene IGHV3-21 was used for VH-CDR grafting. The human VK germline genes IGKV7-3, IGKV1-39, and IGKV4-1 were used for VK-CDR grafting, and generated three versions of humanized 23H8 VK chains. By combining the humanized VH and the three humanized VKs, we generated mAbs hu23H8v1, hu23H8v2 and hu23H8v3, respectively. These three humanized mAbs had similar affinity to hAREG as the chimeric 23H8 (murine variable regions and human IgG1 constant regions), indicating that grafting of the VK-CDRs of 23H8 to the three different human VK germline backbones were all successful. A couple of additional mutations were introduced into the humanized mAbs to remove the potential undesired post-translational modifications or immunogenicity, and three more variants were generated, hu23H8v4, -v5 and -v6. The CDRs of these mAbs were compared to the murine antibody as shown in Table 7. The nucleotide sequences and the amino acid sequences of VH and VL CDRs of these mAbs were shown in Table 8. The SPR-determined binding affinities of these mAbs to hAREG were listed in Tables 9-10.

TABLE 7
Comparison of CDRs among
different versions of mAb 23H8

| | HCDR1 | HCDR2 | HCDR3 |
|---|---|---|---|
| | (SEQ ID No.) | (SEQ ID No.) | (SEQ ID No.) |
| m23H8 | SYAMS(1) | TISTGGSHTYYP | HGYLLYDGYYEW |
| | | DSVKG(13) | YFDV(14) |
| hu23H8v1 | SYAMS(1) | TISTGGSHTYYP | HGYLLYDGYYEW |
| | | DSVKG(13) | YFDY(136) |
| hu23H8v2 | SYAMS(1) | TISTGGSHTYYP | HGYLLYDGYYEW |
| | | DSVKG(13) | YFDY(136) |
| hu23H8v3 | SYAMS(1) | TISTGGSHTYYP | HGYLLYDGYYEW |
| | | DSVKG(13) | YFDY(136) |
| hu23H8v4 | SYAMS(1) | TISTGGSHTYYP | HGYLLYEGYYEW |
| | | ESVKG(15) | YFDY(16) |
| hu23H8v5 | SYAMS(1) | TISTGGSHTYYP | HGYLLYEGYYEW |
| | | ESVKG(15) | YFDY(16) |
| hu23H8v6 | SYAMS(1) | TISTGGSHTYYP | HGYLLYEGYYEW |
| | | ESVKG(15) | YFDY(16) |
| | LCDR1 | LCDR2 | LCDR3 |
| | (SEQ ID No.) | (SEQ ID No.) | (SEQ ID No.) |
| m23H8 | KASQSVDYDGHS | AASNLES(37) | QQSTEDPPYT |
| | FLN(36) | | (38) |
| hu23H8v1 | RASESVDYDGHS | AASNKDT(40) | QQSTEDPPYT |
| | FIN(39) | | (38) |
| hu23H8v2 | RASQSVDYDGHS | AASNLQS(42) | QQSTEDPPYT |
| | FLN(41) | | (38) |
| hu23H8v3 | KSSQSVDYDGHS | AASNRES(44) | QQSTEDPPYT |
| | FLN(43) | | (38) |
| hu23H8v4 | RASESVDYDGHS | AASNKDT(40) | QQSTEDPPYT |
| | FIN(39) | | (38) |
| hu23H8v5 | RASQSVDYEGHS | AASNLQS(42) | QQSTENPPYT |
| | FLN(45) | | (46) |
| hu23H8v6 | KSSQSVDYEGHS | AASNRES(44) | QQSTENPPYT |
| | FLN(47) | | (46) |

The differences between Abs were underlined.
CDRs are defined using Kabat system.

TABLE 8

| Name | VH | VH | VL | VL |
|---|---|---|---|---|
| of the | (nucleotide | (amino acid | (nucleotide | (amino acid |
| Antibody | sequence) | sequence) | sequence) | sequence) |
| 23H8 | SEQ ID | SEQ ID | SEQ ID | SEQ ID |
| | NO: 106 | NO: 65 | NO: 107 | NO: 78 |
| hu23H8v1 | SEQ ID | SEQ ID | SEQ ID | SEQ ID |
| | NO: 108 | NO: 66 | NO: 109 | NO: 79 |
| hu23H8v2 | SEQ ID | SEQ ID | SEQ ID | SEQ ID |
| | NO: 108 | NO: 66 | NO: 110 | NO: 80 |
| hu23H8v3 | SEQ ID | SEQ ID | SEQ ID | SEQ ID |
| | NO: 108 | NO: 66 | NO: 111 | NO: 81 |
| hu23H8v4 | SEQ ID | SEQ ID | SEQ ID | SEQ ID |
| | NO: 112 | NO: 67 | NO: 109 | NO: 79 |
| hu23H8v5 | SEQ ID | SEQ ID | SEQ ID | SEQ ID |
| | NO: 112 | NO: 67 | NO: 113 | NO: 82 |
| hu23H8v6 | SEQ ID | SEQ ID | SEQ ID | SEQ ID |
| | NO: 112 | NO: 67 | NO: 114 | NO: 83 |

TABLE 9

| | hAREG-EGFd | | |
|---|---|---|---|
| mAbs | Kₐ (M⁻¹, s⁻¹) | K_{off} (s) | K_{D} (M) |
| ch23H8 | 4.18 × 10⁵ | 3.13 × 10⁻⁴ | 7.50 × 10⁻¹⁰ |
| hu23H8V1 | 1.05 × 10⁶ | 2.14 × 10⁻⁴ | 2.05 × 10⁻¹⁰ |
| hu23H8V2 | 1.02 × 10⁶ | 2.49 × 10⁻⁴ | 2.44 × 10⁻¹⁰ |
| hu23H8V3 | 1.20 × 10⁶ | 2.13 × 10⁻⁴ | 1.78 × 10⁻¹⁰ |
| hu23H8V4 | 8.21 × 10⁵ | 2.76 × 10⁻⁴ | 3.36 × 10⁻¹⁰ |
| hu23H8V5 | 6.28 × 10⁵ | 3.48 × 10⁻⁴ | 5.65 × 10⁻¹⁰ |
| hu23H8V6 | 8.03 × 10⁵ | 2.46 × 10⁻⁴ | 3.09 × 10⁻¹⁰ |

TABLE 10

| | hAREG-98aa | | |
|---|---|---|---|
| mAbs | Kₐ (M⁻¹, s⁻¹) | K_{off} (s) | K_{D} (M) |
| hu23H8V5 | 1.26 × 10⁷ | 8.63 × 10⁻⁴ | 6.96 × 10⁻¹⁰ |
| hu23H8V6 | 1.11 × 10⁵ | 5.95 × 10⁻⁴ | 5.37 × 10⁻¹⁰ |

Humanization of 1H9

Similar to humanization of 23H8, the human VH germline gene IGHV3-21 was used for VH-CDR grafting; the human VK germline gene IGKV7-3, IGKV1-39, and IGKV4-1 were used for VK-CDR grafting. The CDRs of these mAbs were compared to the murine antibody as shown in Table 11. The nucleotide sequences and the amino acid sequences of VH and VL CDRs of these mAbs were shown in Table 12. The SPR-determined binding affinities of these mAbs to hAREG were listed in Table 13.

TABLE 11
Comparison of CDRs among
different versions of mAb 1H9

| | HCDR1 | HCDR2 | HCDR3 |
|---|---|---|---|
| | (SEQ ID No.) | (SEQ ID No.) | (SEQ ID No.) |
| m1H9 | GYPMS(17) | TISTGARHTYYP | HEGLRRGKYHCI |
| | | DSVKG(18) | MDY(19) |
| hu1H9v1-5 | GYPMS(17) | TISTGARHTYYP | HEGLRRGKYHSI |
| | | DSVKG(18) | MDY(20) |
| | LCDR1 | LCDR2 | LCDR3 |
| | (SEQ ID No.) | (SEQ ID No.) | (SEQ ID No.) |
| m1H9(15) | KASQSIDYDGDS | AASNLES(37) | HQCNEDPYM |
| | FLN(48) | | (49) |
| hu1H9v1(16) | RASESVDYDGDS | AASNKDT(40) | HQSNEDPYM |
| | FIN(50) | | (51) |
| hu1H9v2(17) | RASESVDYDGDS | AASNKDT(40) | HQSNEDPYL |
| | FIN(50) | | (52) |
| hu1H9v3(18) | RASESVDYDGDS | AASNKDT(40) | HQSNEDPYV |
| | FIN(50) | | (53) |
| hu1H9v4(19) | RASQSIDYDGDS | AASNLQS(42) | QQSNEDPYV |
| | FLN(54) | | (55) |
| hu1H9v5(20) | KSSQSIDYDGDS | AASNRES(44) | QQSNEDPYV |
| | FLN(56) | | (55) |

The differences between Abs were underlined.
CDRs are defined using Kabat system.

TABLE 12

| Name | VH | VH | VL | VL |
|---|---|---|---|---|
| of the | (nucleotide | (amino acid | (nucleotide | (amino acid |
| Antibody | sequence) | sequence) | sequence) | sequence) |
| 1H9 | SEQ ID | SEQ ID | SEQ ID | SEQ ID |
| | NO: 115 | NO: 68 | NO: 116 | NO: 84 |
| hu1H9v1 | SEQ ID | SEQ ID | SEQ ID | SEQ ID |
| | NO: 117 | NO: 69 | NO: 118 | NO: 85 |
| hu1H9v2 | SEQ ID | SEQ ID | SEQ ID | SEQ ID |
| | NO: 117 | NO: 69 | NO: 119 | NO: 86 |
| hu1H9v3 | SEQ ID | SEQ ID | SEQ ID | SEQ ID |
| | NO: 117 | NO: 69 | NO: 120 | NO: 87 |
| hu1H9v4 | SEQ ID | SEQ ID | SEQ ID | SEQ ID |
| | NO: 117 | NO: 69 | NO: 121 | NO: 88 |
| hu1H9v5 | SEQ ID | SEQ ID | SEQ ID | SEQ ID |
| | NO: 117 | NO: 69 | NO: 122 | NO: 89 |

TABLE 13

| | hAREG-EGFd | | |
|---|---|---|---|
| mAbs | Kₐ (M⁻¹, s⁻¹) | K_{off} (s) | K_{D} (M) |
| ch1H9 | 3.58 × 10⁵ | 4.67 × 10⁻⁴ | 1.31 × 10⁻¹⁰ |
| hu1H9v1 | 2.91 × 10⁵ | 2.33 × 10⁻⁴ | 7.99 × 10⁻¹⁰ |
| hu1H9v2 | 3.01 × 10⁵ | 2.29 × 10⁻⁴ | 7.62 × 10⁻¹⁰ |
| hu1H9v3 | 2.67 × 10⁵ | 2.59 × 10⁻⁴ | 9.69 × 10⁻¹⁰ |
| hu1H9v4 | 2.43 × 10⁵ | 1.84 × 10⁻⁴ | 7.58 × 10⁻¹⁰ |
| hu1H9v5 | 2.54 × 10⁵ | 2.06 × 10⁻⁴ | 8.11 × 10⁻¹⁰ |

### Example 3. Activity Analysis of Anti-AREG mAbs

1. Preparation of AREG Proteins for In Vitro Activity Analysis of Anti-hAREG Antibodies

Human or mouse EGFR extracellular domain (ECD) encoding cDNA fused with a His6 and an Avi tag at the C-terminus, was co-transfected with a plasmid encoding BirA-hFc for biotinylation in 293F cells. 72 hours after transfection, the cell culture supernatant was harvested for purification of the hEGFR ECD His6-Avi-biotin fusion protein or mEGFR ECD His6-Avi-biotin fusion protein (hEFGR-ECD, mEGFR-ECD) by Protein A affinity chromatography.

Human or mouse AREG EGF domain with four additional residues (DLLA) at the C-terminus, was expressed in 293F cells as an mFc-fusion protein. 72 hours after transfection, the cell culture supernatant was harvested for purification of the hAREG-EGFd-DLLA-mFc (hAREG-DLLA) or mAREG-EGFd-DLLA-mFc (mAREG-DLLA) fusion proteins by Protein A affinity chromatography.

2. Inhibition of hAREG for Binding to EGFR Analyzed by Competition ELISA

Briefly, streptavidin (Sigma, 5 μg/mL) were coated in U-bottom 96-well plates, 100 nM biotinylated hEGFR-ECD or mEGFR-ECD in 100 μL per well were then captured onto the plates. Different antibodies at serial diluted concentrations were mixed with 5 nM hAREG-DLLA or 50 nM mAREG-DLLA protein and added to the ELISA plate. The binding of hAREG-DLLA to hEGFR-ECD or the binding of mAREG-DLLA to mEGFR-ECD was detected by HRP-conjugated mouse anti-mouse IgG Fc antibody (Thermo Fisher).

3. Inhibition of EGFR Receptor Phosphorylation

A431 (a human epidermoid carcinoma cell line) cells were serum-starved for one hour, and were subsequently either treated with hAREG-DLLA (2.5 nM) alone or treated with the mixtures of hAREG and anti-AREG antibodies for one hour. Approximately 1-2×10⁵ cells/well in 6-well plates were used in each treatment. The treated cells were washed with PBS twice and then lysed on ice using RIPA buffer. The cell lysates were then subjected to SDS-PAGE and followed by Western blotting. The phosphorylated form of EGFR (tyrosine 1068) and total EGFR were detected using an anti-phosphotyrosine mAb (Abcam, EP774Y) and a rabbit polyclonal antibody (Cell Signaling technology, #2232), respectively. An anti-α-Tubulin mAb (clone B-5-1-2, Sigma-Aldrich) was used to detect alpha Tubulin expression in the cell lysates, and served as a loading control for Western blotting analysis.

4. Epitope Mapping

To identify the epitopes of our anti-AREG mAbs, five amino acids that are different and have distinct physical properties between hAREG-EGFd and mAREG-EGFd were chosen for mutagenesis. Five hAREG-EGFd variants were generated by changing each amino acid at five different sites of hAREG-EGFd to the counterpart amino acid of mAREG-EGFd. In addition, two mAREG-EGFd variants were generated by changing each amino acid at two different sites of mAREG-EGFd to the counterpart amino acid of hAREG-EGFd. These variants were then examined for binding with anti-AREG mAbs using SPR (Biacore T200).

5. Results

Anti-AREG mAbs Block AREG Binding to EGFR

It has been previously shown that the addition of four amino acids (DLLA) to the C-terminal of EGF domain of human AREG greatly improved the biological activity of recombinant expressed EGF domain (Thompson et al., 1996). To facilitate the competition ELISA assay, we used the hAREG-EGFd-DLLA-mFc (hAREG-DLLA) or mAREG-EGFd-DLLA-mFc (mAREG-DLLA) as the ligands for binding to EGFR in the assay. The results showed that E1H3L4, P7 and hu9C12v4 all competed with mAREG-DLLA for binding to mEGFR-ECD. hu9C12v4 showed the best activity among these three mAbs. hu9C12v4, hu9C12v6, hu23H8v5, hu23H8v6 and hu1H9v3 in their hIgG1 forms were also tested in the competition ELISA, and they all showed potent activity in competition with hAREG-DLLA for binding to hEGFR-ECD with subnanomolar IC50.

In addition, we also tested two previously reported antibodies, huPAR34 (U.S. patent application No. 2004/0210040) and AR558 (US20170002068A1) in human IgG1 forms. These two antibodies also showed potent activity in competition with hAREG-DLLA for binding to hEGFR-ECD.

Anti-AREG mAbs Inhibit EGFR Phosphorylation

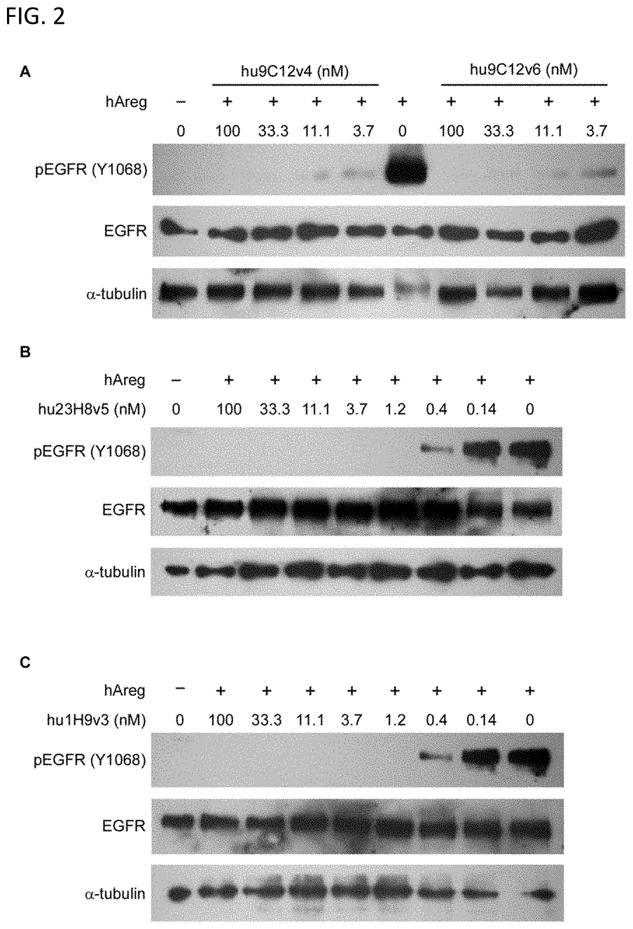

Anti-AREG mAbs were tested for their inhibition activities against EGFR phosphorylation in hEGFR-expressing epidermoid carcinoma cells, A431. Low concentrations of the antibodies were sufficient for blocking AREG-induced phosphorylation of EGFR of A431 cells. 1.2 nM of 23H8 or 1H9 completely blocked hAREG-induced phosphorylation of EGFR. 9C12 showed relatively weaker blocking activity than 23H8 and 1H9 (FIG. 2).

Epitope Mapping

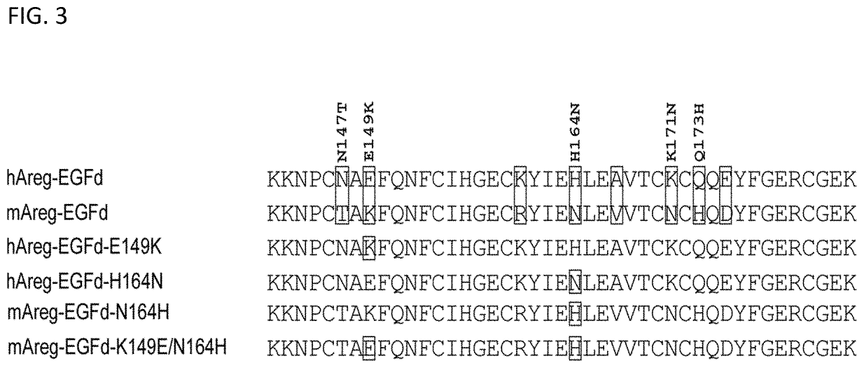

To identify the epitopes of our anti-AREG mAbs, five hAREG-EGFd variants were generated by changing each amino acid at five different sites of hAREG-EGFd to the counterpart amino acid of mAREG-EGFd (FIG. 3). The variants were then examined for binding with anti-AREG mAbs using SPR (Biacore T200). Two amino acids (Glu149 and His164) were identified as critical epitope residues for the binding of the mAb to hAREG. As revealed by Biacore analysis, for hu9C12v4, hu9C12v6, hu23H8 and hu1H9 mAbs, the hAREG-H164N variant completely lost binding activity to mAbs, hAREG-E149K variant had slightly reduced binding activity, and other three hAREG variants had no effect on the binding of the mAbs, demonstrating that His164 is the most critical epitope residue for the binding of our anti-AREG mAbs. For huPAR34, E149K and H164N variants had reduced binding activity, other three residue changes have no or minor effect. For AR558, the E149K variant completely lost binding activity, other four residue changes have no or minor effect on the binding of hAREG to AR558, and indicating Glu149 is the most critical epitope residue for AR558.

In addition, using two mAREG variants, we found that mAREG-K149E/N164H (using hAREG numbering) variant gained full binding affinity for the anti-hAREG antibodies that have no or very weak cross-reactivity to mAREG; the mAREG-N164H variant gained partial binding ability to the antibodies. These results indicate that the amino acids, Lys149 and Asn164 in mAREG, are residues responsible for lack of cross reactivity of the mAbs (hu9C12v6, hu23H8, hu1H9, huPAR34 and AR558) to mAREG.

### Example 4. Animal Study

1. Establishing Animal Models

Cdc42 AT2 Null Mice are Generated by Knocking Out Cdc42 Gene Specifically in Alveolar Type II Cells (AT2 Cells)

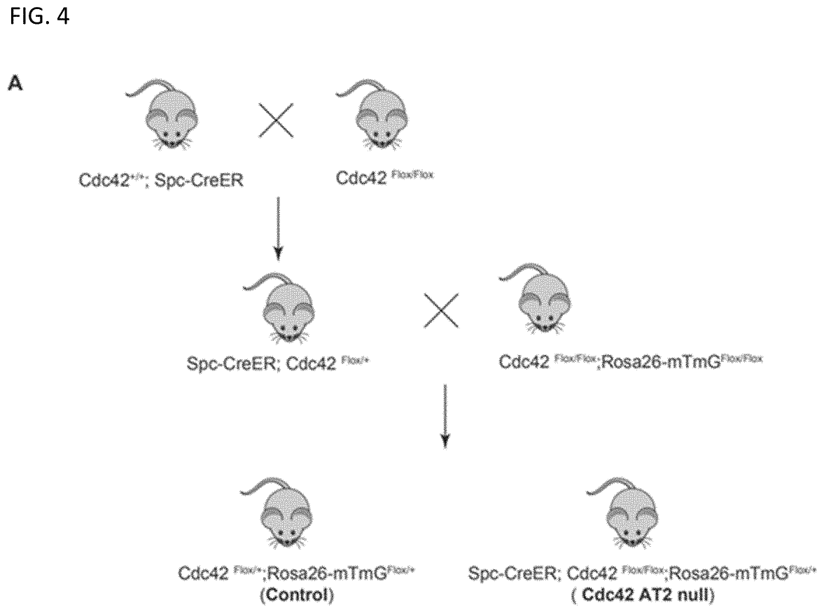

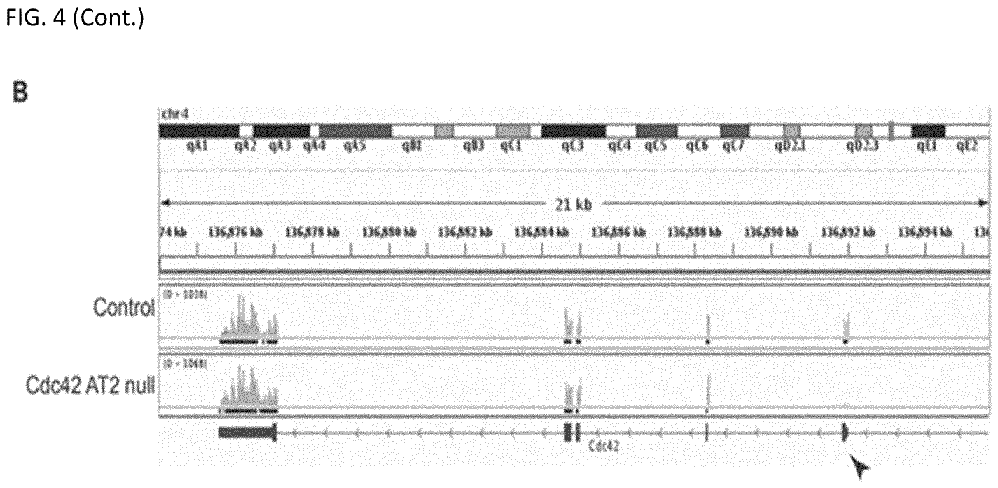

In order to specifically delete Cdc42 gene in AT2 cells, mice carrying a Spc-CreER knock-in allele are crossed with Cdc42 floxed (Cdc42^{flox/flox}) mice (FIG. 4A). In Cdc42^{flox/flox} mice, the exon 2 of Cdc42 gene, which contains the translation initiation exon of Cdc42 gene, is flanked by two loxp sites. In Spc-CreER; Cdc42^{flox/flox} mice the exon 2 of Cdc42 gene, exon 2 of Cdc42 gene is specifically deleted in AT2 cells by Cre/loxp-mediated recombination after tamoxifen treatment (FIG. 4B). Spc-CreER; Cdc42^{flox/flox} mice are named as Cdc42 AT2 null mice. The fragments of Cdc42 DNA sequence before and after deleting the exon2 of the Cdc42 gene are shown as follows. All these mice were maintained in the animal facility in specific pathogen free conditions.

The Cdc42 sequence before deleting the exon2 of the Cdc42 gene is shown in SEQ ID NO: 133. The Cdc42 sequence after deleting the exon2 of the Cdc42 gene is shown in SEQ ID NO: 134.

Lungs of Cdc42 AT2 Null Mice Develop Progressive Fibrotic Changes after PNX Treatment

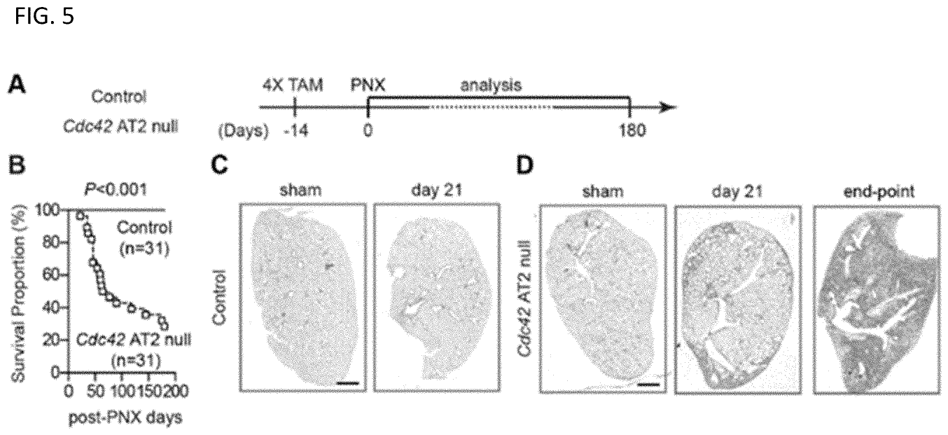

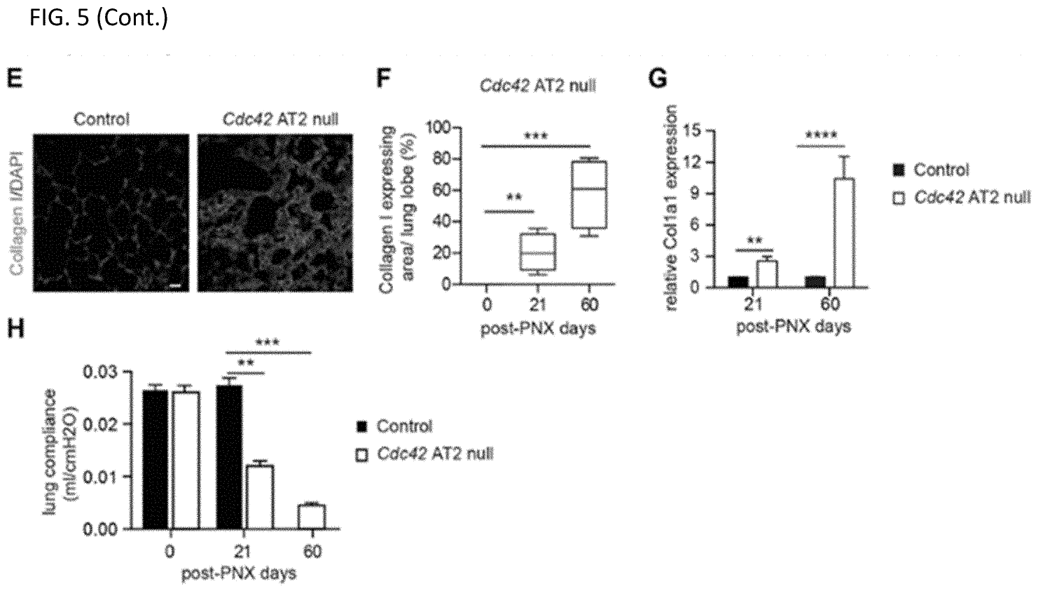

Left lung lobe resection (peumonectomy, PNX) on Cdc42 AT2 null mice and control mice were performed. The lungs of Cdc42 AT2 null mice and control mice at different time points after PNX treatment were analyzed (FIG. 5A). We found that some Cdc42 AT2 null mice showed significant weight loss and increased respiration rates after post-PNX day 21. Indeed, fully 50% of PNX-treated Cdc42 AT2 null mice reached the predefined health-status criteria for endpoint euthanization by post-PNX day 60 (FIG. 5B), and more than 70% of PNX-treated Cdc42 AT2 null mice (n=33) reached their endpoints by post-PNX day 180 (FIG. 5B). H&E staining shows lungs of sham-treated and PNX-treated control mice do not shown fibrotic changes (FIG. 5C). H&E staining shows that the entire lung lobes of PNX-treated Cdc42 AT2 null mice at endpoints have dense fibrotic changes (FIG. 5D).

The lungs of Cdc42 AT2 null mice start to show fibrotic changes at post-PNX day 21. The Cdc42 AT2 null lungs have shown dense fibrotic changes at the edge of lungs (FIG. 5D). H&E staining shows that histological changes of the fibrotic region of Cdc42 AT2 null lungs recapitulate the histological changes of human IPF lungs.

Lungs collected from Control and Cdc42 AT2 null mice at post-PNX day 21 were stained with an anti-Collagen I antibody (FIG. 5E). Much stronger immunofluorescence signals for Collagen I are detected in the dense fibrotic regions of lungs of Cdc42 AT2 null mice as compared with control lungs. The area of dense Collagen I in lungs of Cdc42 AT2 null mice gradually increases from post-PNX day 21 to post-PNX day 60 (FIG. 5F). qPCR analysis showed that the Collagen I mRNA expression levels increased gradually from post-PNX day 21 to post-PNX day 60 in the lungs of Cdc42 AT2 null mice (FIG. 5G). Respiratory function analysis shows that the lung compliance gradually decreased in Cdc42 AT2 null mice from post-PNX day 21 to post-PNX day 60 (FIG. 5H). *P<0.05, ***P<0.001; ****P<0.0001, Student's t test.

This is the first mouse model that can highly mimic the pathogenesis and progression of IPF. Therefore, hereafter called IPF-like lung fibrosis mouse model. Using this animal model, we identified that AREG is a potential therapeutic target for pulmonary fibrosis.

Bleomycin-Induced Lung Fibrosis Mouse Model

Bleomycin-induced pulmonary fibrosis is a common experimental study model of human lung fibrosis. Wild type FVB/N mice (Charles River) in each group were intratracheally instilled with a single dose of BLM (1U/1 KG body weight, H20055883, Hai Zheng Pfizer Inc). The BLM-treated mice in all groups were closed monitored at different time points after the bleomycin administration.

This is an animal model that can recapitulate acute lung injury-induced lung fibrosis, such as post-pneumonia lung fibrosis or ILD (interstitial lung diseases). Bleomycin induces lung injury via oxidant-mediated DNA breaks, leading to alveolar epithelial cell death (1-3 days after injury) and acute inflammatory responses 3-9 days after injury). And lung fibrosis occurs in the lungs at 10-21 days post-injury.

We adopted our IPF-like mouse model and bleomycin-induced lung fibrosis mouse model to explore the therapeutic effects of our AREG antibodies. Furthermore, we also compared the therapeutic effects of two drugs, Nintedanib and Pirfenidone, in order to comprehensively evaluate the potential therapeutic effects of AREG antibodies with existing FDA approved drugs.

2. The Animal Study Design and Analysis of Treatments of Pulmonary Fibrosis in the Mouse Models

1) IPF-like lung fibrosis mouse model: Three-month-old male Cdc42 AT2 null mice with a similar body weight (˜30 g) were selected for the experiments. The mice were injected with tamoxifen intraperitoneally (dosage: 75 mg/kg) four times every other day. Two weeks after the last injection, the mice were treated with PNX. Post-PNX day 14 is the time-point when fibrosis starts. At post-PNX 14 days, PNX-treated mice were weighed and proceed to the treatments.

2) Bleomycin-induced lung fibrosis mouse model: Three-month-old male FVB/N mice with a similar body weight (˜30 g) were selected for the bleomycin treatment. Specifically, an endotracheal cannula was inserted into the trachea of anesthetized mice before delivering bleomycin solution (dosage: 1U/kg). Then one day after the bleomycin delivery, mice were weighed and proceed to treatments.

3) Treatment groups: Mice were divided into different groups: Control, anti-AREG antibody, Nintedanib, and Pirfenidone groups. All mice in each group are age matched and body weight matched. For control groups, mice were treated with an isotype-match control antibody. Control antibody or anti-AREG antibodies were given intraperitoneally at 10-15 mg/kg, every 5 days. In addition, mice in the control group were treated with 0.5% sodium methylcellulose sodium solution via oral gavage once a day. Mice in the Nintedanib group were treated with Nintedanib via oral gavage once a day (60 mg/kg). Mice in the Pirfenidone treatment group were treated with Pirfenidone (100 mg/kg) via oral gavage once a day. Mice in the Nintedanib group and Pirfenidone group were also treated with PBS solution intraperitoneally every five days.

4) Animal study

a) The body weight of the mice in all groups was monitored every other day. The general health condition of mice in all groups is closely monitored twice a day.

b) The humane endpoint is defined by the loss of overall body weight (30% of their initial body weight).

Animal studies were conducted under the approved Institutional Animal Care and Use Committee protocols. Lung tissues were collected at the endpoint of the study. The hydroxyproline content in each mouse lung was measured by a hydroxyproline kit (Sigma, Cat #MAK008). Lung fibrosis scale was evaluated using the histology analysis. Lung tissues were fixed by 4% PFA, sectioned and proceed for H&E staining. The final histological fibrosis scores were assigned by analyzing various fields of the lung.

3. Results

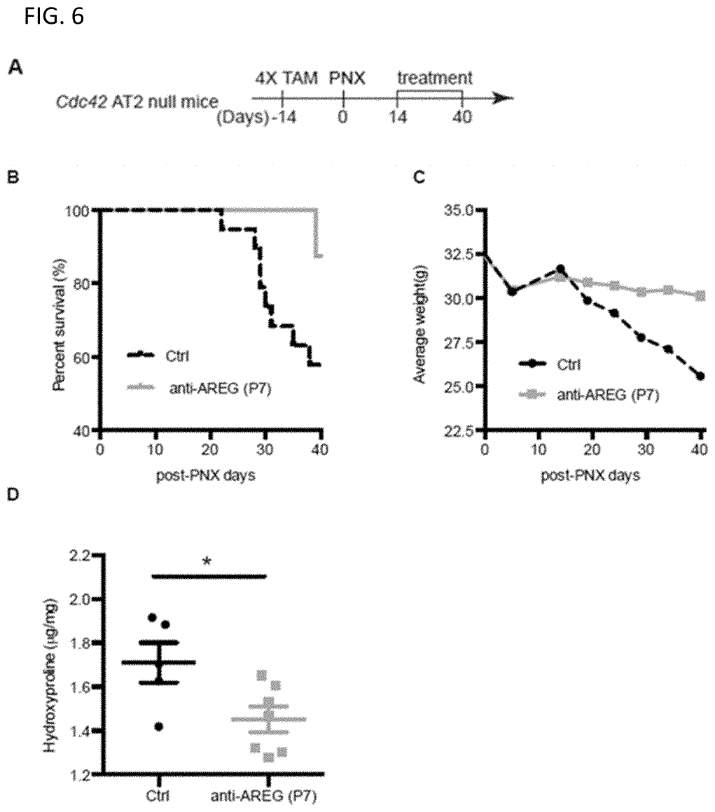

1) Anti-AREG antibody (P7): Our results show the anti-AREG (P7) antibody can significantly slow the weight loss of the Cdc42 AT2 null and can prolong the survival time of Cdc42 AT2 null mice (FIG. 6A-6C). In addition, the anti-AREG antibody (P7) can significantly reduce the content of hydroxyproline in the lungs of the Cdc42 AT2 null mice (FIG. 6D). FIG. 6A shows outline scheme of the treatment and sampling procedure, FIG. 6B shows that the anti-AREG antibody (P7) can prolong the survival time of Cdc42 AT2 null mice, FIG. 6C shows that the anti-AREG antibody (P7) can significantly slow down the weight loss of the Cdc42 AT2 null mice, and FIG. 6D shows that the anti-AREG antibody (P7) can significantly reduce the content of hydroxyproline in the lungs of Cdc42 AT2 null mice as compared with mice treated with the blank antibody (*, P<0.05, Student's t test).

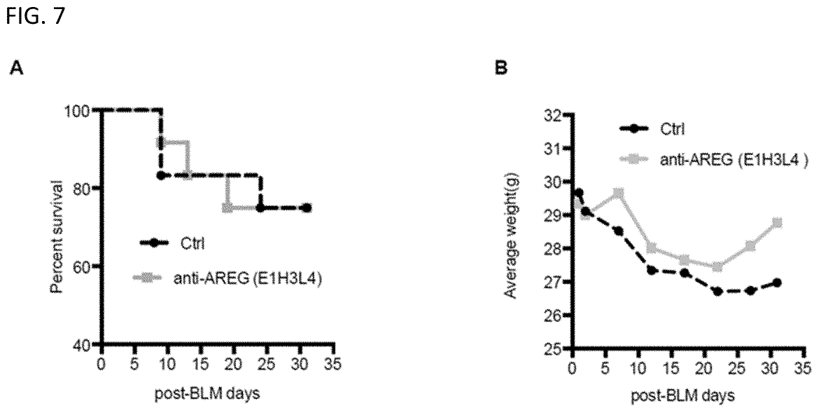

2) Anti-AREG antibody (E1H3L4): Our results show that the anti-AREG antibody (E1H3L4) can accelerate the resolution of fibrosis and promote the weight recovery in the bleomycin-treated mice (FIG. 7B). FIG. 7A shows that the survival rates of mice treated with the blank antibody and mice treated with the anti-AREG antibody (E1H3L4) are not significantly different in the bleomycin-induced lung fibrosis mouse model. However, FIG. 7B shows that the mice in the anti-AREG antibody (E1H3L4) treatment group recovered better than the mice in the blank antibody treatment group.

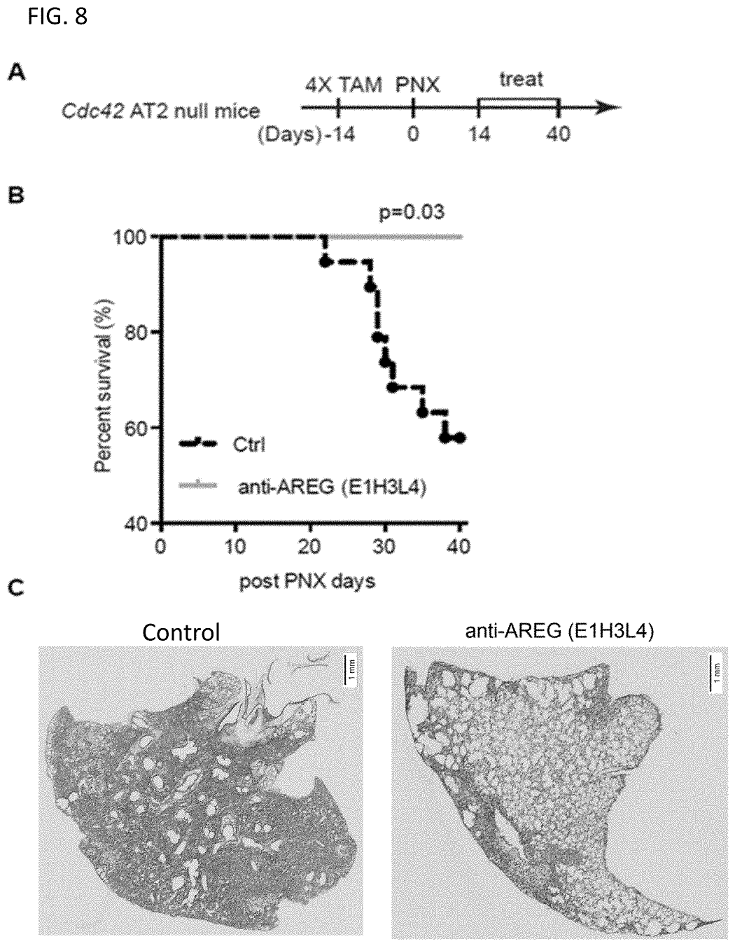

In addition, the anti-AREG (E1H3L4) antibody treatment can significantly prolong the survival time of Cdc42 AT2 null mice (FIG. 8A-8B). The H&E staining analysis showed that the area of mouse lung fibrosis in the anti-AREG (E1H3L4) treatment group was significantly reduced in the lungs of Cdc42 AT2 null mice (FIG. 8C). FIG. 8A shows the outline scheme of the treatment and sampling procedure. FIG. 8B shows that the anti-AREG antibody (E1H3L4) can significantly prolong the survival time of Cdc42 AT2 null mice, and FIG. 8C shows that the lung fibrosis was significantly reduced in the mice of the anti-AREG antibody (E1H3L4) treatment group as compared with the mice in the control group through the H&E staining analysis.

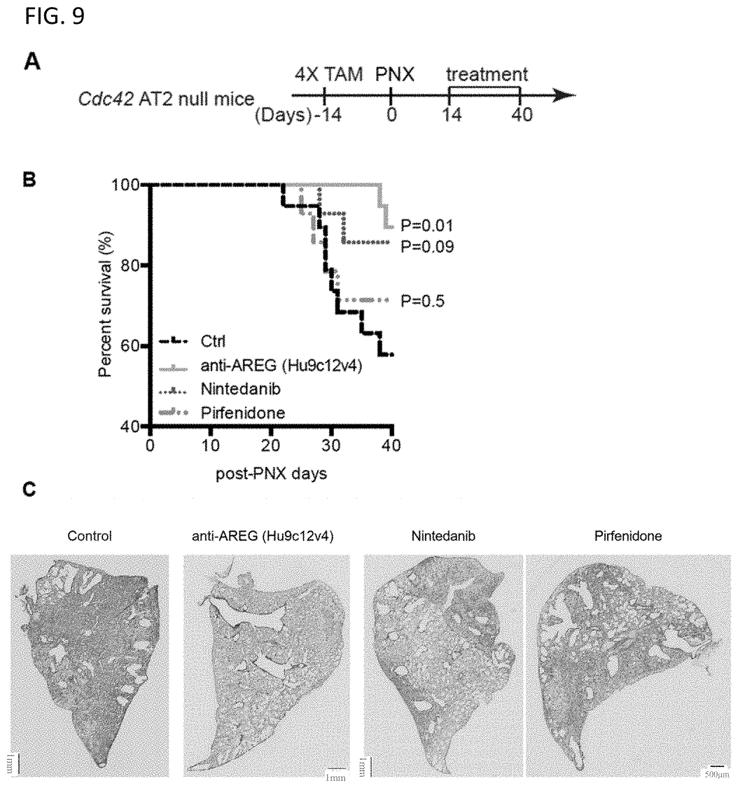

3) Anti-AREG antibody (hu9C12v4): Our results show that the anti-AREG (hu9C12v4) can significantly prolong the survival time of Cdc42 AT2 null mice (FIG. 9A-9B), whereas ninetadnib and pirfenidone do not significantly prolong the survival time of Cdc42 AT2 null mice. The H&E staining analysis showed that the area of mouse lung fibrosis in the anti-AREG antibody (hu9C12v4) treatment group was significantly reduced in the lungs of the Cdc42 AT2 null mice (FIG. 9C). Specifically, FIG. 9A shows the outline scheme of the treatment and sampling procedure, FIG. 9B shows that the anti-AREG antibody (hu9C12v4) treatment can significantly prolong the survival time of Cdc42 AT2 null mice, and FIG. 9C shows that the lung fibrosis was significantly reduced in the mice of the anti-AREG antibody (hu9C12v4) treatment group as compared with the mice in the control, Nintedanib, and Pirfenidone groups through the H&E staining analysis.

Taking together, these results demonstrate that our anti-AREG monoclonal antibodies are effective for treating lung fibrosis.

### REFERENCE

1 Barkauskas, C. E., and Noble, P. W. (2014). Cellular mechanisms of tissue fibrosis. 7. New insights into the cellular mechanisms of pulmonary fibrosis. American journal of physiology Cell physiology 306, C987-996.
2 Li, D., He, W., Liu, X., Zheng, S., Qi, Y., Li, H., Mao, F., Liu, J., Sun, Y., Pan, L., et al. (2017). A potent human neutralizing antibody Fc-dependently reduces established HBV infections. eLife 6: e26738.
3 Steele, M. P., and Schwartz, D. A. (2013). Molecular mechanisms in progressive idiopathic pulmonary fibrosis. Annual review of medicine 64, 265-276.
4 Thompson, S. A., Harris, A., Hoang, D., Ferrer, M., and Johnson, G. R. (1996). COOH-terminal extended recombinant amphiregulin with bioactivity comparable with naturally derived growth factor. The Journal of biological chemistry 271, 17927-17931.

What is claimed is:

1. An isolated anti-AREG antibody or fragment thereof, having the ability of inhibiting fibrosis, preferably, the fibrosis is renal fibrosis, hepatic fibrosis, pulmonary fibrosis, more preferably, IPF.

2. The anti-AREG antibody or fragment thereof of claim 1, which is capable of binding to AREG, preferably, binding to human AREG.

3. The anti-AREG antibody or fragment thereof of claim 1, which is a human anti-AREG antibody, or a murine anti-AREG antibody, or a humanized anti-AREG antibody, or a chimeric anti-AREG antibody, preferably, is a human monoclonal antibody (mAb), murine mAb, humanized mAb, or chimeric mAb, or which preferably is Fab fragment or F(ab)₂ fragment.

4. The anti-AREG antibody or fragment thereof of claim 1, which binds to AREG with high affinity, preferably, with a dissociation constant (KD) of less than about 10 nM, preferably, less than 1 nM, 0.1 nM, or 0.01 nM, preferably, in the range of 1×10⁻⁸-1×10⁻¹¹, more preferably, in the range of 1×10⁻⁹-1×10⁻¹¹.

5. The anti-AREG antibody or fragment thereof of claim 1, which is capable of binding to soluble forms of AREG, preferably, is capable of binding to EGF-like domain of soluble forms of AREG.

6. The anti-AREG antibody or fragment thereof of claim 1, which is capable of binding to residues 101-184 of the human pro-AREG, and/or residues 171-184 of the human pro-AREG, and/or residues 94-177 of the murine pro-AREG, and/or residues 135-177 of the murine pro-AREG.

7. The anti-AREG antibody or fragment thereof of claim 1, which is capable of binding at least one, two, three, four or five amino acids within residues 101-184 of pro-AREG shown by any one of SEQ ID NOs: 123-132, preferably, within residues 142-184 of human pro-AREG shown by any one of SEQ ID NOs: 123-132.

8. The anti-AREG antibody or fragment thereof of claim 1, which is capable of interacting with Glu149 and/or His164 of human pro-AREG.

9. The anti-AREG antibody or fragment thereof of claim 1, which comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, and HCDR3 are selected from the group consisting of:
(1) HCDR1 shown by SEQ ID NO: 1, HCDR2 shown by SEQ ID NO: 2, HCDR3 shown by SEQ ID NO: 3;
(2) HCDR1 shown by SEQ ID NO: 1, HCDR2 shown by SEQ ID NO: 2, HCDR3 shown by SEQ ID NO: 4;
(3) HCDR1 shown by SEQ ID NO: 5, HCDR2 shown by SEQ ID NO: 2, HCDR3 shown by SEQ ID NO: 6;
(4) HCDR1 shown by SEQ ID NO: 7, HCDR2 shown by SEQ ID NO: 8, HCDR3 shown by SEQ ID NO: 9;
(5) HCDR1 shown by SEQ ID NO: 7, HCDR2 shown by SEQ ID NO: 10, HCDR3 shown by SEQ ID NO: 9;
(6) HCDR1 shown by SEQ ID NO: 7, HCDR2 shown by SEQ ID NO: 8, HCDR3 shown by SEQ ID NO: 11;
(7) HCDR1 shown by SEQ ID NO: 7, HCDR2 shown by SEQ ID NO: 8, HCDR3 shown by SEQ ID NO: 12;
(8) HCDR1 shown by SEQ ID NO: 1, HCDR2 shown by SEQ ID NO: 13, HCDR3 shown by SEQ ID NO: 14;
(9) HCDR1 shown by SEQ ID NO: 1, HCDR2 shown by SEQ ID NO: 15, HCDR3 shown by SEQ ID NO: 16;
(10) HCDR1 shown by SEQ ID NO: 17, HCDR2 shown by SEQ ID NO: 18, HCDR3 shown by SEQ ID NO: 19;
(11) HCDR1 shown by SEQ ID NO: 17, HCDR2 shown by SEQ ID NO: 18, HCDR3 shown by SEQ ID NO: 20;
(12) HCDR1 shown by SEQ ID NO: 1, HCDR2 shown by SEQ ID NO: 13, HCDR3 shown by SEQ ID NO: 136; and
(13) HCDR1, HCDR2, HCDR3 as shown in (1)-(12), but at least one of which includes one, two, three, four or five amino acids addition, deletion, conservative amino acid substitution or the combinations thereof; and
LCDR1, LCDR2, and LCDR3 are selected from the group consisting of:
(1) LCDR1 shown by SEQ ID NO: 21, LCDR2 shown by SEQ ID NO: 22, LCDR3 shown by SEQ ID NO: 23;
(2) LCDR1 shown by SEQ ID NO: 21, LCDR2 shown by SEQ ID NO: 22, LCDR3 shown by SEQ ID NO: 24;
(3) LCDR1 shown by SEQ ID NO: 25, LCDR2 shown by SEQ ID NO: 26, LCDR3 shown by SEQ ID NO: 27;
(4) LCDR1 shown by SEQ ID NO: 28, LCDR2 shown by SEQ ID NO: 29, LCDR3 shown by SEQ ID NO: 30;
(5) LCDR1 shown by SEQ ID NO: 31, LCDR2 shown by SEQ ID NO: 32, LCDR3 shown by SEQ ID NO: 30;
(6) LCDR1 shown by SEQ ID NO: 33, LCDR2 shown by SEQ ID NO: 34, LCDR3 shown by SEQ ID NO: 30;
(7) LCDR1 shown by SEQ ID NO: 35, LCDR2 shown by SEQ ID NO: 34, LCDR3 shown by SEQ ID NO: 30;
(8) LCDR1 shown by SEQ ID NO: 36, LCDR2 shown by SEQ ID NO: 37, LCDR3 shown by SEQ ID NO: 38;
(9) LCDR1 shown by SEQ ID NO: 39, LCDR2 shown by SEQ ID NO: 40, LCDR3 shown by SEQ ID NO: 38;
(10) LCDR1 shown by SEQ ID NO: 41, LCDR2 shown by SEQ ID NO: 42, LCDR3 shown by SEQ ID NO: 38;
(11) LCDR1 shown by SEQ ID NO: 43, LCDR2 shown by SEQ ID NO: 44, LCDR3 shown by SEQ ID NO: 38;
(12) LCDR1 shown by SEQ ID NO: 39, LCDR2 shown by SEQ ID NO: 40, LCDR3 shown by SEQ ID NO: 38;
(13) LCDR1 shown by SEQ ID NO: 45, LCDR2 shown by SEQ ID NO: 42, LCDR3 shown by SEQ ID NO: 46;
(14) LCDR1 shown by SEQ ID NO: 47, LCDR2 shown by SEQ ID NO: 44, LCDR3 shown by SEQ ID NO: 46;
(15) LCDR1 shown by SEQ ID NO: 48, LCDR2 shown by SEQ ID NO: 37, LCDR3 shown by SEQ ID NO: 49;
(16) LCDR1 shown by SEQ ID NO: 50, LCDR2 shown by SEQ ID NO: 40, LCDR3 shown by SEQ ID NO: 51;
(17) LCDR1 shown by SEQ ID NO: 50, LCDR2 shown by SEQ ID NO: 40, LCDR3 shown by SEQ ID NO: 52;
(18) LCDR1 shown by SEQ ID NO: 50, LCDR2 shown by SEQ ID NO: 40, LCDR3 shown by SEQ ID NO: 53;
(19) LCDR1 shown by SEQ ID NO: 54, LCDR2 shown by SEQ ID NO: 42, LCDR3 shown by SEQ ID NO: 55;
(20) LCDR1 shown by SEQ ID NO: 56, LCDR2 shown by SEQ ID NO: 44, LCDR3 shown by SEQ ID NO: 55; and
(21) LCDR1, LCDR2, LCDR3 as shown in (1)-(20), but at least one of which includes one, two, three, four or five amino acids addition, deletion, conservative amino acid substitution or the combinations thereof.

10. The anti-AREG antibody or fragment thereof of claim 1, which comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, HCDR3, LCDR1, LCDR2, and LCDR3 are selected from the group consisting of:
(1) HCDR1 shown by SEQ ID NO: 1, HCDR2 shown by SEQ ID NO: 2, HCDR3 shown by SEQ ID NO: 3, LCDR1 shown by SEQ ID NO: 21, LCDR2 shown by SEQ ID NO: 22, LCDR3 shown by SEQ ID NO: 23;
(2) HCDR1 shown by SEQ ID NO: 1, HCDR2 shown by SEQ ID NO: 2, HCDR3 shown by SEQ ID NO: 4, LCDR1 shown by SEQ ID NO: 21, LCDR2 shown by SEQ ID NO: 22, LCDR3 shown by SEQ ID NO: 24;
(3) HCDR1 shown by SEQ ID NO: 5, HCDR2 shown by SEQ ID NO: 2, HCDR3 shown by SEQ ID NO: 6, LCDR1 shown by SEQ ID NO: 25, LCDR2 shown by SEQ ID NO: 26, LCDR3 shown by SEQ ID NO: 27;
(4) HCDR1 shown by SEQ ID NO: 7, HCDR2 shown by SEQ ID NO: 8, HCDR3 shown by SEQ ID NO: 9, LCDR1 shown by SEQ ID NO: 28, LCDR2 shown by SEQ ID NO: 29, LCDR3 shown by SEQ ID NO: 30;
(5) HCDR1 shown by SEQ ID NO: 7, HCDR2 shown by SEQ ID NO: 10, HCDR3 shown by SEQ ID NO: 9, LCDR1 shown by SEQ ID NO: 31, LCDR2 shown by SEQ ID NO: 32, LCDR3 shown by SEQ ID NO: 30;
(6) HCDR1 shown by SEQ ID NO: 7, HCDR2 shown by SEQ ID NO: 8, HCDR3 shown by SEQ ID NO: 11, LCDR1 shown by SEQ ID NO: 33, LCDR2 shown by SEQ ID NO: 34, LCDR3 shown by SEQ ID NO: 30;
(7) HCDR1 shown by SEQ ID NO: 7, HCDR2 shown by SEQ ID NO: 8, HCDR3 shown by SEQ ID NO: 12, LCDR1 shown by SEQ ID NO: 35, LCDR2 shown by SEQ ID NO: 34, LCDR3 shown by SEQ ID NO: 30;
(8) HCDR1 shown by SEQ ID NO: 1, HCDR2 shown by SEQ ID NO: 13, HCDR3 shown by SEQ ID NO: 14, LCDR1 shown by SEQ ID NO: 36, LCDR2 shown by SEQ ID NO: 37, LCDR3 shown by SEQ ID NO: 38;
(9) HCDR1 shown by SEQ ID NO: 1, HCDR2 shown by SEQ ID NO: 13, HCDR3 shown by SEQ ID NO: 136, LCDR1 shown by SEQ ID NO: 39, LCDR2 shown by SEQ ID NO: 40, LCDR3 shown by SEQ ID NO: 38;
(10) HCDR1 shown by SEQ ID NO: 1, HCDR2 shown by SEQ ID NO: 13, HCDR3 shown by SEQ ID NO: 136, LCDR1 shown by SEQ ID NO: 41, LCDR2 shown by SEQ ID NO: 42, LCDR3 shown by SEQ ID NO: 38;
(11) HCDR1 shown by SEQ ID NO: 1, HCDR2 shown by SEQ ID NO: 13, HCDR3 shown by SEQ ID NO: 136, LCDR1 shown by SEQ ID NO: 43, LCDR2 shown by SEQ ID NO: 44, LCDR3 shown by SEQ ID NO: 38;
(12) HCDR1 shown by SEQ ID NO: 1, HCDR2 shown by SEQ ID NO: 15, HCDR3 shown by SEQ ID NO: 16, LCDR1 shown by SEQ ID NO: 39, LCDR2 shown by SEQ ID NO: 40, LCDR3 shown by SEQ ID NO: 38;
(13) HCDR1 shown by SEQ ID NO: 1, HCDR2 shown by SEQ ID NO: 15, HCDR3 shown by SEQ ID NO: 16, LCDR1 shown by SEQ ID NO: 45, LCDR2 shown by SEQ ID NO: 42, LCDR3 shown by SEQ ID NO: 46;
(14) HCDR1 shown by SEQ ID NO: 1, HCDR2 shown by SEQ ID NO: 15, HCDR3 shown by SEQ ID NO: 16, LCDR1 shown by SEQ ID NO: 47, LCDR2 shown by SEQ ID NO: 44, LCDR3 SHOWN BY SEQ ID NO: 46;
(15) HCDR1 shown by SEQ ID NO: 17, HCDR2 shown by SEQ ID NO: 18, HCDR3 shown by SEQ ID NO: 19, LCDR1 shown by SEQ ID NO: 48, LCDR2 shown by SEQ ID NO: 37, LCDR3 shown by SEQ ID NO: 49;
(16) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 51;
(17) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 52;
(18) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 53;
(19) HCDR1 shown by SEQ ID NO: 17, HCDR2 shown by SEQ ID NO: 18, HCDR3 shown by SEQ ID NO: 20, LCDR1 shown by SEQ ID NO: 54, LCDR2 shown by SEQ ID NO: 42, LCDR3 shown by SEQ ID NO: 55;
(20) HCDR1 shown by SEQ ID NO: 17, HCDR2 shown by SEQ ID NO: 18, HCDR3 shown by SEQ ID NO: 20, LCDR1 shown by SEQ ID NO: 56, LCDR2 shown by SEQ ID NO: 44, LCDR3 shown by SEQ ID NO: 55; and
(21) HCDR1, HCDR2, HCDR3, LCDR1, LCDR2, LCDR3 as shown in (1)-(20), but at least one of which includes one, two, three, four or five amino acids addition, deletion, conservative amino acid substitution or the combinations thereof.

11. The anti-AREG antibody or fragment thereof of claim 1, which comprises a heavy chain variable region, and a light chain variable region, wherein the heavy chain variable region has the amino acid sequence selected from the group consisting of SEQ ID NOs: 57-69, and an amino acid sequence having at least 95% sequence identity to any one of SEQ ID NOs: 57-69, and retaining the activity of epitope-binding,
wherein the light chain variable region has the amino acid sequence selected from the group consisting of SEQ ID NOs: 70-89, and an amino acid sequence having at least 95% sequence identity to any one of SEQ ID NOs: 70-89, and retaining the activity of epitope-binding.

12. The anti-AREG antibody or fragment thereof of claim 1, which comprises a heavy chain variable region, and a light chain variable region, wherein the heavy chain variable region and the light chain variable region have the amino acid sequences selected from the group consisting of:
(1) SEQ ID NO: 57 and SEQ ID NO: 70;
(2) SEQ ID NO: 58 and SEQ ID NO: 71;
(3) SEQ ID NO: 59 and SEQ ID NO: 72;
(4) SEQ ID NO: 60 and SEQ ID NO: 73;
(5) SEQ ID NO: 61 and SEQ ID NO: 74;
(6) SEQ ID NO: 62 and SEQ ID NO: 75;
(7) SEQ ID NO: 63 and SEQ ID NO: 76;
(8) SEQ ID NO: 64 and SEQ ID NO: 77;
(9) SEQ ID NO: 65 and SEQ ID NO: 78;
(10) SEQ ID NO: 66 and SEQ ID NO: 79;
(11) SEQ ID NO: 66 and SEQ ID NO: 80;
(12) SEQ ID NO: 66 and SEQ ID NO: 81;
(13) SEQ ID NO: 67 and SEQ ID NO: 79;
(14) SEQ ID NO: 67 and SEQ ID NO: 82;
(15) SEQ ID NO: 67 and SEQ ID NO: 83;
(16) SEQ ID NO: 68 and SEQ ID NO: 84;
(17) SEQ ID NO: 69 and SEQ ID NO: 85;
(18) SEQ ID NO: 69 and SEQ ID NO: 86;
(19) SEQ ID NO: 69 and SEQ ID NO: 87;
(20) SEQ ID NO: 69 and SEQ ID NO: 88;
(21) SEQ ID NO: 69 and SEQ ID NO: 89; and
(22) two amino acid sequences having at least 95% sequence identity to any one of (1)-(21) respectively, and retaining the activity of epitope-binding.

13. The anti-AREG antibody or fragment thereof of claim 1, which is an isotype of IgG, IgM, IgA, IgE or IgD, preferably, an isotype of IgG1, IgG2, IgG3, or IgG4.

14. An isolated polynucleotide or a nucleic acid encoding the anti-AREG antibody or fragment thereof according to claim 1,
wherein the polynucleotide or nucleic acid encodes the entire heavy chain variable region, or the entire light chain variable region, or the both on the same polynucleotide or on separate polynucleotides; or
wherein the polynucleotide or nucleic acid encodes portions of heavy chain variable region, or the light chain variable region, or the both on the same polynucleotide or on separate polynucleotides.

15. The isolated polynucleotide or a nucleic acid of claim 14, which comprises the DNA sequence encoding the heavy chain variable region shown by any one of sequences SEQ ID NOs: 90, 92, 94, 96, 98, 100, 102, 104, 106, 108, 112, 115, and 117, and/or the DNA sequence encoding the light chain variable region shown by any one of sequences SEQ ID NOs: 91, 93, 95, 97, 99, 101, 103, 105, 107, 109, 110, 111, 113, 114, 116, 118, 119, 120, 121, and 122.

16. An isolated cell, or vector comprising one or more polynucleotide encoding the anti-AREG antibody or fragment thereof according to claim 1.

17. A composition comprising the anti-AREG antibody or fragment thereof according to claim 1, and a pharmaceutical acceptable carrier.

18. A method for treating a disorder, in which AREG is overexpressed, upregulated or activated, in a subject, comprising administering to the subject the anti-AREG antibody or fragment thereof according to claim 1, wherein the disorder is a fibrotic disease including but not limited to renal fibrosis, hepatic fibrosis, pulmonary fibrosis, in particular, IPF.

19. A method for diagnosing a disorder, in which AREG is overexpressed, upregulated or activated, comprising exposing a sample from a subject suspected of suffering from the disorder to the anti-AREG antibody or fragment thereof according to claim 1, and determining binding of the anti-AREG antibody or fragment thereof to the sample, wherein the disorder is a fibrotic disease, including but not limited to renal fibrosis, hepatic fibrosis, pulmonary fibrosis, in particular, IPF.

20. An isolated AREG protein, having an amino acid sequence shown in any one of SEQ ID NOs: 123-132, or an amino acid sequence that is at least 85% identical to any one of SEQ ID NOs: 123-132.

21. The isolated AREG protein of claim 20, which is an epitope for producing the anti-AREG antibody or fragment wherein the AREG antibody or fragment has the ability of inhibiting fibrosis, preferably, the fibrosis is renal fibrosis, hepatic fibrosis, pulmonary fibrosis, more preferably, IPF.

22. The isolated AREG protein of claim 20, which has the amino acid Glu149 and/or His164.


SEQUENCE LISTING (136 sequences)

SEQ ID NO: 1 - protein, 5 aa, synthetic construct
  REGION 1..5: note = Synthetic polypeptide
SYAMS

SEQ ID NO: 2 - protein, 17 aa, synthetic construct
  REGION 1..17: note = Synthetic polypeptide
AISGSGGSTYYADSVKG

SEQ ID NO: 3 - protein, 11 aa, synthetic construct
  REGION 1..11: note = Synthetic polypeptide
PTSRYSYGYDY

SEQ ID NO: 4 - protein, 11 aa, synthetic construct
  REGION 1..11: note = Synthetic polypeptide
PTSRYSYSYNN

SEQ ID NO: 5 - protein, 5 aa, synthetic construct
  REGION 1..5: note = Synthetic polypeptide
SHAMS

SEQ ID NO: 6 - protein, 7 aa, synthetic construct
  REGION 1..7: note = Synthetic polypeptide
VDTKFDP

SEQ ID NO: 7 - protein, 5 aa, synthetic construct
  REGION 1..5: note = Synthetic polypeptide
SYPMS

SEQ ID NO: 8 - protein, 17 aa, synthetic construct
  REGION 1..17: note = Synthetic polypeptide
TISTGGTYTYYPDSVKG

SEQ ID NO: 9 - protein, 15 aa, synthetic construct
  REGION 1..15: note = Synthetic polypeptide
QGPIYYGNYYYAMDY

SEQ ID NO: 10 - protein, 17 aa, synthetic construct
  REGION 1..17: note = Synthetic polypeptide
TISTGGRYTYYPDSVKG

SEQ ID NO: 11 - protein, 15 aa, synthetic construct
  REGION 1..15: note = Synthetic polypeptide
QGPILRKNYYYGMDV

SEQ ID NO: 12 - protein, 15 aa, synthetic construct
  REGION 1..15: note = Synthetic polypeptide
QGPIYYGNYYYGMDV

SEQ ID NO: 13 - protein, 17 aa, synthetic construct
  REGION 1..17: note = Synthetic polypeptide
TISTGGSHTYYPDSVKG

SEQ ID NO: 14 - protein, 16 aa, synthetic construct
  REGION 1..16: note = Synthetic polypeptide
HGYLLYDGYYEWYFDV

SEQ ID NO: 15 - protein, 17 aa, synthetic construct
  REGION 1..17: note = Synthetic polypeptide
TISTGGSHTYYPESVKG

SEQ ID NO: 16 - protein, 16 aa, synthetic construct
  REGION 1..16: note = Synthetic polypeptide
HGYLLYEGYYEWYFDY

SEQ ID NO: 17 - protein, 5 aa, synthetic construct
  REGION 1..5: note = Synthetic polypeptide
GYPMS

SEQ ID NO: 18 - protein, 17 aa, synthetic construct
  REGION 1..17: note = Synthetic polypeptide
TISTGARHTYYPDSVKG

SEQ ID NO: 19 - protein, 15 aa, synthetic construct
  REGION 1..15: note = Synthetic polypeptide
HEGLRRGKYHCIMDY

SEQ ID NO: 20 - protein, 15 aa, synthetic construct
  REGION 1..15: note = Synthetic polypeptide
HEGLRRGKYHSIMDY

SEQ ID NO: 21 - protein, 13 aa, synthetic construct
  REGION 1..13: note = Synthetic polypeptide
TGNSNNVGDQGAV

SEQ ID NO: 22 - protein, 7 aa, synthetic construct
  REGION 1..7: note = Synthetic polypeptide
RNNNRPS

SEQ ID NO: 23 - protein, 11 aa, synthetic construct
  REGION 1..11: note = Synthetic polypeptide
STWDSGLNSVV

SEQ ID NO: 24 - protein, 11 aa, synthetic construct
  REGION 1..11: note = Synthetic polypeptide
STWDKNNKSVV

SEQ ID NO: 25 - protein, 13 aa, synthetic construct
  REGION 1..13: note = Synthetic polypeptide
SGSSSNIGSNTVN

SEQ ID NO: 26 - protein, 7 aa, synthetic construct
  REGION 1..7: note = Synthetic polypeptide
SNNQRPS

SEQ ID NO: 27 - protein, 11 aa, synthetic construct
  REGION 1..11: note = Synthetic polypeptide
EVWDDSLNGPV

SEQ ID NO: 28 - protein, 16 aa, synthetic construct
  REGION 1..16: note = Synthetic polypeptide
RSSQSLVHSDGNTYLH

SEQ ID NO: 29 - protein, 7 aa, synthetic construct
  REGION 1..7: note = Synthetic polypeptide
KVSNRFS

SEQ ID NO: 30 - protein, 9 aa, synthetic construct
  REGION 1..9: note = Synthetic polypeptide
SQSTHVPYT

SEQ ID NO: 31 - protein, 16 aa, synthetic construct
  REGION 1..16: note = Synthetic polypeptide
RSSQSLVDGEDGTYLN

SEQ ID NO: 32 - protein, 7 aa, synthetic construct
  REGION 1..7: note = Synthetic polypeptide
KVSERFD

SEQ ID NO: 33 - protein, 16 aa, synthetic construct
  REGION 1..16: note = Synthetic polypeptide
RSSQSLVDGQDGTYLH

SEQ ID NO: 34 - protein, 7 aa, synthetic construct
  REGION 1..7: note = Synthetic polypeptide
KVSNRFD

SEQ ID NO: 35 - protein, 16 aa, synthetic construct
  REGION 1..16: note = Synthetic polypeptide
RSSQSLVNQEGETYLH

SEQ ID NO: 36 - protein, 15 aa, synthetic construct
  REGION 1..15: note = Synthetic polypeptide
KASQSVDYDGHSFLN

SEQ ID NO: 37 - protein, 7 aa, synthetic construct
  REGION 1..7: note = Synthetic polypeptide
AASNLES

SEQ ID NO: 38 - protein, 10 aa, synthetic construct
  REGION 1..10: note = Synthetic polypeptide
QQSTEDPPYT

SEQ ID NO: 39 - protein, 15 aa, synthetic construct
  REGION 1..15: note = Synthetic polypeptide
RASESVDYDGHSFIN

SEQ ID NO: 40 - protein, 7 aa, synthetic construct
  REGION 1..7: note = Synthetic polypeptide
AASNKDT

SEQ ID NO: 41 - protein, 15 aa, synthetic construct
  REGION 1..15: note = Synthetic polypeptide
RASQSVDYDGHSFLN

SEQ ID NO: 42 - protein, 7 aa, synthetic construct
  REGION 1..7: note = Synthetic polypeptide
AASNLQS

SEQ ID NO: 43 - protein, 15 aa, synthetic construct
  REGION 1..15: note = Synthetic polypeptide
KSSQSVDYDGHSFLN

SEQ ID NO: 44 - protein, 7 aa, synthetic construct
  REGION 1..7: note = Synthetic polypeptide
AASNRES

SEQ ID NO: 45 - protein, 15 aa, synthetic construct
  REGION 1..15: note = Synthetic polypeptide
RASQSVDYEGHSFLN

SEQ ID NO: 46 - protein, 10 aa, synthetic construct
  REGION 1..10: note = Synthetic polypeptide
QQSTENPPYT

SEQ ID NO: 47 - protein, 15 aa, synthetic construct
  REGION 1..15: note = Synthetic polypeptide
KSSQSVDYEGHSFLN

SEQ ID NO: 48 - protein, 15 aa, synthetic construct
  REGION 1..15: note = Synthetic polypeptide
KASQSIDYDGDSFLN

SEQ ID NO: 49 - protein, 9 aa, synthetic construct
  REGION 1..9: note = Synthetic polypeptide
HQCNEDPYM

SEQ ID NO: 50 - protein, 15 aa, synthetic construct
  REGION 1..15: note = Synthetic polypeptide
RASESVDYDGDSFIN

SEQ ID NO: 51 - protein, 9 aa, synthetic construct
  REGION 1..9: note = Synthetic polypeptide
HQSNEDPYM

SEQ ID NO: 52 - protein, 9 aa, synthetic construct
  REGION 1..9: note = Synthetic polypeptide
HQSNEDPYL

SEQ ID NO: 53 - protein, 9 aa, synthetic construct
  REGION 1..9: note = Synthetic polypeptide
HQSNEDPYV

SEQ ID NO: 54 - protein, 15 aa, synthetic construct
  REGION 1..15: note = Synthetic polypeptide
RASQSIDYDGDSFLN

SEQ ID NO: 55 - protein, 9 aa, synthetic construct
  REGION 1..9: note = Synthetic polypeptide
QQSNEDPYV

SEQ ID NO: 56 - protein, 15 aa, synthetic construct
  REGION 1..15: note = Synthetic polypeptide
KSSQSIDYDGDSFLN

SEQ ID NO: 57 - protein, 120 aa, synthetic construct
  REGION 1..120: note = Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPTSRYSYGYDYWGQGTLVTVSS

SEQ ID NO: 58 - protein, 120 aa, synthetic construct
  REGION 1..120: note = Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPTSRYSYSYNNWGQGTLVTVSS

SEQ ID NO: 59 - protein, 116 aa, synthetic construct
  REGION 1..116: note = Synthetic polypeptide
EVQLVQSGGGVVQPGRSLRLSCAASGFIFSSHAMSWVRQAPGKGLEWVSAISGSGGSTYYADSVKGRFTISRDNSKNTLYLQMNSLRAEDTAVYYCAKVDTKFDPWGQGTLVTVSS

SEQ ID NO: 60 - protein, 124 aa, synthetic construct
  REGION 1..124: note = Synthetic polypeptide
EVKLVESGGGLVKPGGSLKLSCAASGFTFSSYPMSWVRQTPEKRLEWVATISTGGTYTYYPDSVKGRFTISRDNAKNTLYLQMSSLRSEDTAMYYCARQGPIYYGNYYYAMDYWGQGTSVTVSS

SEQ ID NO: 61 - protein, 124 aa, synthetic construct
  REGION 1..124: note = Synthetic polypeptide
EVQLVESGGGLVKPGGSLRLSCAASGFTFSSYPMSWVRQAPGKGLEWVSTISTGGTYTYYPDSVKGRFTISRDNAKNSLYLQMNSLRAEDTAVYYCARQGPIYYGNYYYAMDYWGQGTTVTVSS

SEQ ID NO: 62 - protein, 124 aa, synthetic construct
  REGION 1..124: note = Synthetic polypeptide
EVQLVESGGGLVKPGGSLRLSCAASGFTFSSYPMSWVRQAPEKGLEWVSTISTGGRYTYYPDSVKGRFTISRDNAKNSLYLQMNSLRAEDTAVYYCARQGPIYYGNYYYAMDYWGQGTTVTVSS

SEQ ID NO: 63 - protein, 124 aa, synthetic construct
  REGION 1..124: note = Synthetic polypeptide
EVQLVESGGGLVKPGGSLRLSCAASGFTFSSYPMSWVRQAPEKGLEWVSTISTGGTYTYYPDSVKGRFTISRDNAKNSLYLQMNSLRAEDTAVYYCARQGPILRKNYYYGMDVWGQGTTVTVSS

SEQ ID NO: 64 - protein, 124 aa, synthetic construct
  REGION 1..124: note = Synthetic polypeptide
EVQLVESGGGLVKPGGSLRLSCAASGFTFSSYPMSWVRQAPEKGLEWVSTISTGGTYTYYPDSVKGRFTISRDNAKNSLYLQMNSLRAEDTAVYYCARQGPIYYGNYYYGMDVWGQGTTVTVSS

SEQ ID NO: 65 - protein, 125 aa, synthetic construct
  REGION 1..125: note = Synthetic polypeptide
EVQLQESGGGLVKPGGSLKLSCAASGFTFNSYAMSWVRQTPEKRLEWVATISTGGSHTYYPDSVKGRFTISRDNAKNTLYLQMSSLRSEDTAMYYCARHGYLLYDGYYEWYFDVWGAGTTVTVSS

SEQ ID NO: 66 - protein, 125 aa, synthetic construct
  REGION 1..125: note = Synthetic polypeptide
EVQLVESGGGLVKPGGSLRLSCAASGFTFNSYAMSWVRQAPGKGLEWVSTISTGGSHTYYPDSVKGRFTISRDNAKNSLYLQMNSLRAEDTAVYYCARHGYLLYDGYYEWYFDYWGQGTLVTVSS

SEQ ID NO: 67 - protein, 125 aa, synthetic construct
  REGION 1..125: note = Synthetic polypeptide
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSS

SEQ ID NO: 68 - protein, 124 aa, synthetic construct
  REGION 1..124: note = Synthetic polypeptide
EVKLMESGGDLVKPGGSLKLSCVASGFTFSGYPMSWVRQTPEKRLEWVATISTGARHTYYPDSVKGRFTISRDNAKNTLYLQMSSLRSEDTAMYYCARHEGLRRGKYHCIMDYWGQGTSVTVSS

SEQ ID NO: 69 - protein, 124 aa, synthetic construct
  REGION 1..124: note = Synthetic polypeptide
EVQLVESGGGLVKPGGSLRLSCAASGFTFSGYPMSWVRQAPGKGLEWVSTISTGARHTYYPDSVKGRFTISRDNAKNSLYLQMNSLRAEDTAVYYCARHEGLRRGKYHSIMDYWGQGTLVTVSS

SEQ ID NO: 70 - protein, 110 aa, synthetic construct
  REGION 1..110: note = Synthetic polypeptide
QAGLTQPPSVSKGLRQTATLTCTGNSNNVGDQGAVWLQQHQGHPPRLLSYRNNNRPSGISERFSASRSGNTASLTITGLQPEDEADYYCSTWDSGLNSVVFGGGTQLTVL

SEQ ID NO: 71 - protein, 110 aa, synthetic construct
  REGION 1..110: note = Synthetic polypeptide
QAVLTQPPSASGTPGQRVTISCTGNSNNVGDQGAVWYQQLPGTAPKLLIYRNNNRPSGVPDRFSGSKSGTSASLAISGLQSEDEADYYCSTWDKNNKSVVFGGGTKLTVL

SEQ ID NO: 72 - protein, 110 aa, synthetic construct
  REGION 1..110: note = Synthetic polypeptide
QSVLTQPPSASGTPGQRVTISCSGSSSNIGSNTVNWYQQLPGTAPKLLIYSNNQRPSGVPDRFSGSKSGTSASLAISGLRSEDEADYYCEVWDDSLNGPVFGGGTKVTVL

SEQ ID NO: 73 - protein, 112 aa, synthetic construct
  REGION 1..112: note = Synthetic polypeptide
DIVMTQPPLSLPVSLGDQASISCRSSQSLVHSDGNTYLHWYLQRPGQSPKLLIYKVSNRFSGVPDRFSGSGSGTDFTLKISRVEAEDLGVYFCSQSTHVPYTFGGGTKLEIK

SEQ ID NO: 74 - protein, 112 aa, synthetic construct
  REGION 1..112: note = Synthetic polypeptide
DIVMTQSPLSLPVTLGQPASISCRSSQSLVHSDGNTYLHWYQQRPGQSPKLLIYKVSNRFSGVPDRFSGSGSGTDFTLKISRVEAEDVGVYYCSQSTHVPYTFGGGTKVEIK

SEQ ID NO: 75 - protein, 112 aa, synthetic construct
  REGION 1..112: note = Synthetic polypeptide
DIVMTQSPLSLPVTLGDPASISCRSSQSLVDGEDGTYLNWFQQRPGQSPKLLIYKVSERFDGVPDRFSGSGSGTDFTLKISRVEAEDVGVYYCSQSTHVPYTFGGGTKVEIK

SEQ ID NO: 76 - protein, 112 aa, synthetic construct
  REGION 1..112: note = Synthetic polypeptide
DVVMTQSPLSLPVTLGDPASISCRSSQSLVDGQDGTYLHWYQQRPGQSPKLLIYKVSNRFDGVPDRFSGSGSGTDFTLKISRVEAEDVGVYYCSQSTHVPYTFGQGTKLEIK

SEQ ID NO: 77 - protein, 112 aa, synthetic construct
  REGION 1..112: note = Synthetic polypeptide
DVVMTQSPLSLPVTLGDPASISCRSSQSLVNQEGETYLHWYQQRPGQSPKLLIYKVSNRFDGVPDRFSGSGSGTDFTLKISRVEAEDVGVYYCSQSTHVPYTFGQGTKLEIK

SEQ ID NO: 78 - protein, 112 aa, synthetic construct
  REGION 1..112: note = Synthetic polypeptide
DIVLTQSPASLAVSLGQRATISCKASQSVDYDGHSFLNWYQQKPGQPPKLLIYAASNLESGLPARFSGSGSGTDFTLNIHPVEEEDAATYYCQQSTEDPPYTFGGGTKLEIK

SEQ ID NO: 79 - protein, 112 aa, synthetic construct
  REGION 1..112: note = Synthetic polypeptide
DIVLTQSPASLAVSPGQRATITCRASESVDYDGHSFINWYQQKPGQPPKLLIYAASNKDTGVPARFSGSGSGTDFTLTINPVEAEDTANYYCQQSTEDPPYTFGQGTKLEIK

SEQ ID NO: 80 - protein, 112 aa, synthetic construct
  REGION 1..112: note = Synthetic polypeptide
DIQMTQSPSSLSASVGDRVTITCRASQSVDYDGHSFLNWYQQKPGKAPKLLIYAASNLQSGVPSRFSGSGSGTDFTLTISSLQPEDFATYYCQQSTEDPPYTFGQGTKLEIK

SEQ ID NO: 81 - protein, 112 aa, synthetic construct
  REGION 1..112: note = Synthetic polypeptide
DIVMTQSPDSLAVSLGERATINCKSSQSVDYDGHSFLNWYQQKPGQPPKLLIYAASNRESGVPDRFSGSGSGTDFTLTISSLQAEDVAVYYCQQSTEDPPYTFGQGTKLEIK

SEQ ID NO: 82 - protein, 112 aa, synthetic construct
  REGION 1..112: note = Synthetic polypeptide
DIQMTQSPSSLSASVGDRVTITCRASQSVDYEGHSFLNWYQQKPGKAPKLLIYAASNLQSGVPSRFSGSGSGTDFTLTISSLQPEDFATYYCQQSTENPPYTFGQGTKLEIK

SEQ ID NO: 83 - protein, 112 aa, synthetic construct
  REGION 1..112: note = Synthetic polypeptide
DIVMTQSPDSLAVSLGERATINCKSSQSVDYEGHSFLNWYQQKPGQPPKLLIYAASNRESGVPDRFSGSGSGTDFTLTISSLQAEDVAVYYCQQSTENPPYTFGQGTKLEIK

SEQ ID NO: 84 - protein, 111 aa, synthetic construct
  REGION 1..111: note = Synthetic polypeptide
DILLTQSPASLAVSLGQRATISCKASQSIDYDGDSFLNWYQQKPGQPPKLLIYAASNLESGIPARFSGSGSGTDFTLNIHPVEEEDAATYYCHQCNEDPYMFGGGTKLEIR

SEQ ID NO: 85 - protein, 111 aa, synthetic construct
  REGION 1..111: note = Synthetic polypeptide
DIVLTQSPASLAVSPGQRATITCRASESVDYDGDSFINWYQQKPGQPPKLLIYAASNKDTGVPARFSGSGSGTDFTLTINPVEAEDTANYYCHQSNEDPYMFGQGTKLEIK

SEQ ID NO: 86 - protein, 111 aa, synthetic construct
  REGION 1..111: note = Synthetic polypeptide
DIVLTQSPASLAVSPGQRATITCRASESVDYDGDSFINWYQQKPGQPPKLLIYAASNKDTGVPARFSGSGSGTDFTLTINPVEAEDTANYYCHQSNEDPYLFGQGTKLEIK

SEQ ID NO: 87 - protein, 111 aa, synthetic construct
  REGION 1..111: note = Synthetic polypeptide
DIVLTQSPASLAVSPGQRATITCRASESVDYDGDSFINWYQQKPGQPPKLLIYAASNKDTGVPARFSGSGSGTDFTLTINPVEAEDTANYYCHQSNEDPYVFGQGTKLEIK

SEQ ID NO: 88 - protein, 111 aa, synthetic construct
  REGION 1..111: note = Synthetic polypeptide
DIQMTQSPSSLSASVGDRVTITCRASQSIDYDGDSFLNWYQQKPGKAPKLLIYAASNLQSGVPSRFSGSGSGTDFTLTISSLQPEDFATYYCQQSNEDPYVFGQGTKLEIK

SEQ ID NO: 89 - protein, 111 aa, synthetic construct
  REGION 1..111: note = Synthetic polypeptide
DIVMTQSPDSLAVSLGERATINCKSSQSIDYDGDSFLNWYQQKPGQPPKLLIYAASNRESGVPDRFSGSGSGTDFTLTISSLQAEDVAVYYCQQSNEDPYVFGQGTKLEIK

SEQ ID NO: 90 - DNA, 360 bp, synthetic construct
  misc_feature 1..360: note = Synthetic polynucleotide
  source 1..360: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaccaacctcaagatacagctatggttacgactactggggccagggaaccctggtcaccgtctcctca

SEQ ID NO: 91 - DNA, 330 bp, synthetic construct
  misc_feature 1..330: note = Synthetic polynucleotide
  source 1..330: mol_type = other DNA
caggcagggctgactcagccaccctcggtgtccaagggcttgagacagaccgccacactcacctgcactgggaacagcaacaatgttggcgaccaaggagcagtttggctgcagcagcaccagggccaccctcccagactcctgtcctacaggaataacaaccggccctcagggatctcagagagattctctgcatccaggtcaggaaacacagcctccctgaccattactggactccagcctgaggacgaggctgactactactgctcaacgtgggacagcggcctcaattctgtggtattcggcggagggacccagctgaccgtccta

SEQ ID NO: 92 - DNA, 361 bp, synthetic construct
  misc_feature 1..361: note = Synthetic polynucleotide
  source 1..361: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaccaacctcaagatacagctacagctacaacaactggggccagggaaccctggtcaccgtctcctcag

SEQ ID NO: 93 - DNA, 330 bp, synthetic construct
  misc_feature 1..330: note = Synthetic polynucleotide
  source 1..330: mol_type = other DNA
caggctgtgctgactcagccaccctcagcgtctgggacccccgggcagagggtcaccatctcttgtactgggaacagcaacaatgttggcgaccaaggagcagtttggtaccagcagctcccaggaacggcccccaaactcctcatctataggaataacaaccggccctcaggggtccctgaccgattctctggctccaagtctggcacctcagcctccctggccatcagtgggctccagtctgaggatgaggctgattattactgttcaacgtgggacaagaacaacaagtctgtggtattcggcggagggaccaagctgaccgtccta

SEQ ID NO: 94 - DNA, 348 bp, synthetic construct
  misc_feature 1..348: note = Synthetic polynucleotide
  source 1..348: mol_type = other DNA
gaagtgcagctggtgcagtctgggggaggcgtggtccagcctgggaggtccctgagactctcctgtgcagcctctggattcatctttagcagccatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtggacaccaaattcgacccctggggccagggaaccctggtcaccgtctcctca

SEQ ID NO: 95 - DNA, 330 bp, synthetic construct
  misc_feature 1..330: note = Synthetic polynucleotide
  source 1..330: mol_type = other DNA
cagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgaagtgtgggatgacagcctgaatggtccggtgttcggcggagggaccaaggtcaccgtccta

SEQ ID NO: 96 - DNA, 372 bp, synthetic construct
  misc_feature 1..372: note = Synthetic polynucleotide
  source 1..372: mol_type = other DNA
gaggtgaagctggtggaatctgggggaggcttagtgaagcctggagggtccctgaaactctcctgtgcagcctctggattcactttcagtagctatcccatgtcttgggttcgccagactccggagaagaggctggagtgggtcgcaaccattagtactggtggtacttacacctactatccagacagtgtgaaggggcgattcaccatctccagagacaatgccaagaacaccctgtacctgcaaatgagcagtctgaggtctgaggacacggccatgtattactgtgcaagacaaggcccgatctactatggtaactactactatgctatggactactggggtcaaggaacctcagtcaccgtctcctca

SEQ ID NO: 97 - DNA, 336 bp, synthetic construct
  misc_feature 1..336: note = Synthetic polynucleotide
  source 1..336: mol_type = other DNA
gacattgtgatgacacaacctccactctccctgcctgtcagtcttggagatcaagcctccatctcttgcagatctagtcagagccttgtacacagtgatggaaacacctatttacattggtacctgcagaggccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaa

SEQ ID NO: 98 - DNA, 372 bp, synthetic construct
  misc_feature 1..372: note = Synthetic polynucleotide
  source 1..372: mol_type = other DNA
gaggtgcagctggtggaatctgggggaggcttagtgaagcctggagggtccctgagactctcctgtgcagcctctggattcactttcagtagctatcccatgtcttgggttcgccaggctccggggaaggggctggagtgggtctcaaccattagtactggtggtacttacacctactatccagacagtgtgaaggggcgattcaccatctccagagacaatgccaagaactccctgtacctgcaaatgaacagtctgagggccgaggacacggccgtgtattactgtgcaagacaaggcccgatctactatggtaactactactatgctatggactactggggtcaaggaaccacggtcaccgtctcctca

SEQ ID NO: 99 - DNA, 336 bp, synthetic construct
  misc_feature 1..336: note = Synthetic polynucleotide
  source 1..336: mol_type = other DNA
gatattgtgatgacacaatctccactctccctgcctgtcacccttggacagccggcctccatctcttgcagatctagtcagagccttgtacacagtgatggaaacacctatttacattggtaccagcagaggccaggccagtctccaaagctcctgatctacaaagtttccaaccgattttctggggtcccagacaggttcagtggcagtggatcagggacagatttcacactcaagatcagcagagtggaggctgaggatgttggagtttattactgctctcaaagtacacatgttccgtacacgttcggaggggggaccaaggtggagatcaaa

SEQ ID NO: 100 - DNA, 372 bp, synthetic construct
  misc_feature 1..372: note = Synthetic polynucleotide
  source 1..372: mol_type = other DNA
gaggtgcagctggtggaatctgggggaggcttagtgaagcctggagggtccctgagactctcctgtgcagcctctggattcactttcagtagctatcccatgtcttgggttcgccaggctccggagaaggggctggagtgggtctcaaccattagtactggtggtcggtacacctactatccagacagtgtgaaggggcgattcaccatctccagagacaatgccaagaactccctgtacctgcaaatgaacagtctgagggccgaggacacggccgtgtattactgtgcaagacaaggcccgatctactatggtaactactactatgctatggactactggggtcaaggaaccacggtcaccgtctcctca

SEQ ID NO: 101 - DNA, 336 bp, synthetic construct
  misc_feature 1..336: note = Synthetic polynucleotide
  source 1..336: mol_type = other DNA
gatattgtgatgacacaatctccactctccctgcctgtcacccttggagacccggcctccatctcttgcagatctagtcagagccttgtagatggggaggatgggacctatttaaactggttccagcagaggccaggccagtctccaaagctcctgatctacaaagtttccgagcgatttgacggggtcccagacaggttcagtggcagtggatcagggacagatttcacactcaagatcagcagagtggaggctgaggatgttggagtttattactgctctcaaagtacacatgttccgtacacgttcggaggggggaccaaggtggagatcaaa

SEQ ID NO: 102 - DNA, 372 bp, synthetic construct
  misc_feature 1..372: note = Synthetic polynucleotide
  source 1..372: mol_type = other DNA
gaggtgcagctggtggaatctgggggaggcttagtgaagcctggagggtccctgagactctcctgtgcagcctctggattcactttcagtagctatcccatgtcttgggttcgccaggctccggagaaggggctggagtgggtctcaaccattagtactggtggtacttacacctactatcccgacagtgtgaaggggcgattcaccatctccagagacaatgccaagaactccctgtacctgcaaatgaacagtctgagggccgaggacacggccgtgtattactgtgcaagacaaggcccgatccttcggaagaattactactatggcatggacgtgtggggtcaaggaaccacggtcaccgtctcctca

SEQ ID NO: 103 - DNA, 336 bp, synthetic construct
  misc_feature 1..336: note = Synthetic polynucleotide
  source 1..336: mol_type = other DNA
gatgttgtgatgactcaatctccactctccctgcctgtcacccttggagacccggcctccatctcttgcagatctagtcagagccttgtagacggccaggacggcacctatttacattggtaccagcagaggccaggccagtctccaaagctcctgatctacaaagtttccaaccgatttgacggggtcccagacaggttcagtggcagtggatcagggacagatttcacactcaagatcagcagagtggaggctgaggatgttggagtttattactgctctcaaagtacacatgttccgtacacgttcggacaggggaccaagctggagatcaaa

SEQ ID NO: 104 - DNA, 372 bp, synthetic construct
  misc_feature 1..372: note = Synthetic polynucleotide
  source 1..372: mol_type = other DNA
gaggtgcagctggtggaatctgggggaggcttagtgaagcctggagggtccctgagactctcctgtgcagcctctggattcactttcagtagctatcccatgtcttgggttcgccaggctccggagaaggggctggagtgggtctcaaccattagtactggtggtacttacacctactatcccgacagtgtgaaggggcgattcaccatctccagagacaatgccaagaactccctgtacctgcaaatgaacagtctgagggccgaggacacggccgtgtattactgtgcaagacaaggcccgatctactatggtaactactactatggcatggacgtgtggggtcaaggaaccacggtcaccgtctcctca

SEQ ID NO: 105 - DNA, 336 bp, synthetic construct
  misc_feature 1..336: note = Synthetic polynucleotide
  source 1..336: mol_type = other DNA
gatgttgtgatgactcaatctccactctccctgcctgtcacccttggagacccggcctccatctcttgcagatctagtcagagccttgtaaatcaggagggtgagacctatttacattggtaccagcagaggccaggccagtctccaaagctcctgatctacaaagtttccaaccgatttgacggggtcccagacaggttcagtggcagtggatcagggacagatttcacactcaagatcagcagagtggaggctgaggatgttggagtttattactgctctcaaagtacacatgttccgtacacgttcggacaggggaccaagctggagatcaaa

SEQ ID NO: 106 - DNA, 375 bp, synthetic construct
  misc_feature 1..375: note = Synthetic polynucleotide
  source 1..375: mol_type = other DNA
gaggtgcagcttcaggagtctgggggaggcttagtgaagcctggagggtccctgaaactctcctgtgcagcctctggcttcactttcaatagctatgccatgtcttgggttcgccagactccggagaagaggctggagtgggtcgcaaccattagtactggtggttctcacacctactatccagacagtgtgaaggggcgattcaccatctccagagacaatgccaagaacaccctatacctgcaaatgagcagtctgaggtctgaggacacggccatgtattactgtgcaagacacggatatctcctctatgatggttactacgaatggtacttcgatgtctggggcgcagggaccacggtcaccgtctcctca

SEQ ID NO: 107 - DNA, 336 bp, synthetic construct
  misc_feature 1..336: note = Synthetic polynucleotide
  source 1..336: mol_type = other DNA
gacattgttctcacccagtctccagcttctttggctgtgtctttagggcagagggccaccatctcctgcaaggccagccaaagtgttgattatgatggtcatagttttctgaactggtaccaacagaaaccaggacagccacccaaactcctcatctatgctgcatccaatctagaatctgggctcccagccaggtttagtggcagtgggtctgggacagacttcaccctcaacatccatcctgtggaggaggaggatgctgcaacctattactgtcagcaaagtactgaggatcctccgtacacgttcggaggggggaccaagctggaaataaaa

SEQ ID NO: 108 - DNA, 375 bp, synthetic construct
  misc_feature 1..375: note = Synthetic polynucleotide
  source 1..375: mol_type = other DNA
gaggtgcagctggtggaatctgggggaggcttagtgaagcctggagggtccctgagactctcctgtgcagcctctggattcactttcaacagctatgccatgtcttgggttcgccaggctccggggaaggggctggagtgggtctcaaccattagtactggtggtagccacacctactatccagacagtgtgaaggggcgattcaccatctccagagacaatgccaagaactccctgtacctgcaaatgaacagtctgagggccgaggacacggccgtgtattactgtgcaagacacggatatctcctctatgatggttactacgaatggtacttcgattactggggtcaaggaaccctggtcaccgtctcctca

SEQ ID NO: 109 - DNA, 336 bp, synthetic construct
  misc_feature 1..336: note = Synthetic polynucleotide
  source 1..336: mol_type = other DNA
gacattgtgctgacccagtctccagcttctttggctgtgtctcccgggcagagggccaccatcacctgccgggccagcgagagtgttgattatgatggtcatagttttatcaactggtaccaacagaaaccaggacagccacccaaactcctcatctatgctgcatccaataaggacaccggggtgccagccaggtttagtggcagtgggtctgggacagacttcaccctcaccatcaaccctgtggaggccgaggataccgcaaactattactgtcagcaaagtactgaggatcctccgtacacgttcggacaggggaccaagctggaaataaaa

SEQ ID NO: 110 - DNA, 336 bp, synthetic construct
  misc_feature 1..336: note = Synthetic polynucleotide
  source 1..336: mol_type = other DNA
gacatccagatgacccagagccccagcagcctgagcgccagcgtgggcgaccgcgtgaccatcacctgccgcgccagccagagcgtggactacgacggccacagcttcctgaactggtaccagcagaagcccggcaaggcccccaagctgctgatctacgccgccagcaacctgcagagcggcgtgcccagccgcttcagcggcagcggcagcggcaccgacttcaccctgaccatcagcagcctgcagcccgaggacttcgccacctactactgccagcagagcaccgaggaccccccctacaccttcggccagggcaccaagctggagatcaag

SEQ ID NO: 111 - DNA, 336 bp, synthetic construct
  misc_feature 1..336: note = Synthetic polynucleotide
  source 1..336: mol_type = other DNA
gacatcgtgatgacccagagccccgacagcctggccgtgagcctgggcgagcgcgccaccatcaactgcaagagcagccagagcgtggactacgacggccacagcttcctgaactggtaccagcagaagcccggccagccccccaagctgctgatctacgccgccagcaaccgcgagagcggcgtgcccgaccgcttcagcggcagcggcagcggcaccgacttcaccctgaccatcagcagcctgcaggccgaggacgtggccgtgtactactgccagcagagcaccgaggaccccccctacaccttcggccagggcaccaagctggagatcaag

SEQ ID NO: 112 - DNA, 375 bp, synthetic construct
  misc_feature 1..375: note = Synthetic polynucleotide
  source 1..375: mol_type = other DNA
gaggtgcagctggtggaatctgggggaggcttagtgaagcctggagggtccctgagactctcctgtgcagcctctggattcactttcagcagctatgccatgtcttgggttcgccaggctccggggaaggggctggagtgggtctcaaccattagtactggtggtagccacacctactatccagagagtgtgaaggggcgattcaccatctccagagacaatgccaagaactccctgtacctgcaaatgaacagtctgagggccgaggacacggccgtgtattactgtgcaagacacggatatctcctctatgagggttactacgaatggtacttcgattactggggtcaaggaaccctggtcaccgtctcctca

SEQ ID NO: 113 - DNA, 336 bp, synthetic construct
  misc_feature 1..336: note = Synthetic polynucleotide
  source 1..336: mol_type = other DNA
gacatccagatgacccagagccccagcagcctgagcgccagcgtgggcgaccgcgtgaccatcacctgccgcgccagccagagcgtggactacgagggccacagcttcctgaactggtaccagcagaagcccggcaaggcccccaagctgctgatctacgccgccagcaacctgcagagcggcgtgcccagccgcttcagcggcagcggcagcggcaccgacttcaccctgaccatcagcagcctgcagcccgaggacttcgccacctactactgccagcagagcaccgagaaccccccctacaccttcggccagggcaccaagctggagatcaag

SEQ ID NO: 114 - DNA, 336 bp, synthetic construct
  misc_feature 1..336: note = Synthetic polynucleotide
  source 1..336: mol_type = other DNA
gacatcgtgatgacccagagccccgacagcctggccgtgagcctgggcgagcgcgccaccatcaactgcaagagcagccagagcgtggactacgagggccacagcttcctgaactggtaccagcagaagcccggccagccccccaagctgctgatctacgccgccagcaaccgcgagagcggcgtgcccgaccgcttcagcggcagcggcagcggcaccgacttcaccctgaccatcagcagcctgcaggccgaggacgtggccgtgtactactgccagcagagcaccgagaaccccccctacaccttcggccagggcaccaagctggagatcaag

SEQ ID NO: 115 - DNA, 372 bp, synthetic construct
  misc_feature 1..372: note = Synthetic polynucleotide
  source 1..372: mol_type = other DNA
gaggtgaagctgatggaatctgggggagacttagtgaagcctggagggtccctgaaactctcctgtgtagcctctggattcactttcagtgggtatcccatgtcttgggttcgccagactccggagaagaggctggagtgggtcgcaaccattagtactggtgctaggcacacctactatccagacagtgtgaaggggcgattcaccatctccagagacaatgccaagaacaccctgtacctgcaaatgagcagtctgaggtctgaggacacggccatgtattactgtgcaagacatgaagggttacgacgagggaaatatcactgtattatggactactggggtcaaggaacctcagtcaccgtctcctca

SEQ ID NO: 116 - DNA, 333 bp, synthetic construct
  misc_feature 1..333: note = Synthetic polynucleotide
  source 1..333: mol_type = other DNA
gatattttgctgactcagtctccagcttctttggctgtgtctctagggcagagggccaccatctcctgcaaggccagtcaaagtattgattatgatggtgatagttttttgaactggtaccaacagaaaccaggacagccacccaaactcctcatctatgctgcatccaatctagaatctgggatcccagccaggtttagtggcagtggatctgggacagacttcaccctcaacatccatcctgtggaggaggaggatgctgcaacctattactgtcatcaatgtaatgaggatccgtacatgttcggaggggggaccaagctggaaataaga

SEQ ID NO: 117 - DNA, 372 bp, synthetic construct
  misc_feature 1..372: note = Synthetic polynucleotide
  source 1..372: mol_type = other DNA
gaggtgcagctggtggaatctgggggaggcttagtgaagcctggagggtccctgagactctcctgtgcagcctctggattcactttcagcggctatcccatgtcttgggttcgccaggctccggggaaggggctggagtgggtctcaaccattagtactggtgccaggcacacctactatccagacagtgtgaaggggcgattcaccatctccagagacaatgccaagaactccctgtacctgcaaatgaacagtctgagggccgaggacacggccgtgtattactgtgcaagacacgaagggttacgacgagggaaatatcacagtattatggattactggggtcaaggaaccctggtcaccgtctcctca

SEQ ID NO: 118 - DNA, 333 bp, synthetic construct
  misc_feature 1..333: note = Synthetic polynucleotide
  source 1..333: mol_type = other DNA
gacattgtgctgacccagtctccagcttctttggctgtgtctcccgggcagagggccaccatcacctgccgggccagcgagagtgttgattatgatggtgacagttttatcaactggtaccaacagaaaccaggacagccacccaaactcctcatctatgctgcatccaataaggacaccggggtgccagccaggtttagtggcagtgggtctgggacagacttcaccctcaccatcaaccctgtggaggccgaggataccgcaaactattactgtcatcaaagtaatgaggatccgtacatgttcggacaggggaccaagctggaaataaaa

SEQ ID NO: 119 - DNA, 333 bp, synthetic construct
  misc_feature 1..333: note = Synthetic polynucleotide
  source 1..333: mol_type = other DNA
gacattgtgctgacccagtctccagcttctttggctgtgtctcccgggcagagggccaccatcacctgccgggccagcgagagtgttgattatgatggtgacagttttatcaactggtaccaacagaaaccaggacagccacccaaactcctcatctatgctgcatccaataaggacaccggggtgccagccaggtttagtggcagtgggtctgggacagacttcaccctcaccatcaaccctgtggaggccgaggataccgcaaactattactgtcatcaaagtaatgaggatccgtacctgttcggacaggggaccaagctggaaataaaa

SEQ ID NO: 120 - DNA, 333 bp, synthetic construct
  misc_feature 1..333: note = Synthetic polynucleotide
  source 1..333: mol_type = other DNA
gacattgtgctgacccagtctccagcttctttggctgtgtctcccgggcagagggccaccatcacctgccgggccagcgagagtgttgattatgatggtgacagttttatcaactggtaccaacagaaaccaggacagccacccaaactcctcatctatgctgcatccaataaggacaccggggtgccagccaggtttagtggcagtgggtctgggacagacttcaccctcaccatcaaccctgtggaggccgaggataccgcaaactattactgtcatcaaagtaatgaggatccgtatgtgttcggacaggggaccaagctggaaataaaa

SEQ ID NO: 121 - DNA, 333 bp, synthetic construct
  misc_feature 1..333: note = Synthetic polynucleotide
  source 1..333: mol_type = other DNA
gacatccagatgacccagagccccagcagcctgagcgccagcgtgggcgaccgcgtgaccatcacctgccgcgccagccagagcatcgactacgacggcgacagcttcctgaactggtaccagcagaagcccggcaaggcccccaagctgctgatctacgccgccagcaacctgcagagcggcgtgcccagccgcttcagcggcagcggcagcggcaccgacttcaccctgaccatcagcagcctgcagcccgaggacttcgccacctactactgccagcagagcaacgaggacccctacgtgttcggccagggcaccaagctggagatcaag

SEQ ID NO: 122 - DNA, 333 bp, synthetic construct
  misc_feature 1..333: note = Synthetic polynucleotide
  source 1..333: mol_type = other DNA
gacatcgtgatgacccagagccccgacagcctggccgtgagcctgggcgagcgcgccaccatcaactgcaagagcagccagagcatcgactacgacggcgacagcttcctgaactggtaccagcagaagcccggccagccccccaagctgctgatctacgccgccagcaaccgcgagagcggcgtgcccgaccgcttcagcggcagcggcagcggcaccgacttcaccctgaccatcagcagcctgcaggccgaggacgtggccgtgtactactgccagcagagcaacgaggacccctacgtgttcggccagggcaccaagctggagatcaag

SEQ ID NO: 123 - protein, 43 aa, Homo sapiens
KKNPCNAEFQNFCIHGECKYIEHLEAVTCKCQQEYFGERCGEK

SEQ ID NO: 124 - protein, 43 aa, Mus musculus
KKNPCTAKFQNFCIHGECRYIENLEVVTCNCHQDYFGERCGEK

SEQ ID NO: 125 - protein, 43 aa, Homo sapiens
KKNPCNAKFQNFCIHGECKYIEHLEAVTCKCQQEYFGERCGEK

SEQ ID NO: 126 - protein, 43 aa, Homo sapiens
KKNPCNAEFQNFCIHGECKYIENLEAVTCKCQQEYFGERCGEK

SEQ ID NO: 127 - protein, 43 aa, Mus musculus
KKNPCTAEFQNFCIHGECRYIENLEVVTCNCHQDYFGERCGEK

SEQ ID NO: 128 - protein, 43 aa, Mus musculus
KKNPCTAEFQNFCIHGECRYIEHLEVVTCNCHQDYFGERCGEK

SEQ ID NO: 129 - protein, 84 aa, Homo sapiens
SVRVEQVVKPPQNKTESENTSDKPKRKKKGGKNGKNRRNRKKKNPCNAEFQNFCIHGECKYIEHLEAVTCKCQQEYFGERCGEK

SEQ ID NO: 130 - protein, 106 aa, Mus musculus
GLNDIFEAQKIEWHEGGGGSGGSVRVEQVIKPKKNKTEGEKSTEKPKRKKKGGKNGKGRRNKKKKNPCTAKFQNFCIHGECRYIENLEVVTCNCHQDYFGERCGEK

SEQ ID NO: 131 - protein, 43 aa, Mus musculus; identical to SEQ ID NO: 124

SEQ ID NO: 132 - protein, 18 aa, synthetic construct
  REGION 1..18: note = Synthetic polypeptide
GSSGKCQQEYFGERCGEK

SEQ ID NO: 133 - DNA, 1128 bp, synthetic construct
  misc_feature 1..1128: note = Synthetic polynucleotide
  source 1..1128: mol_type = other DNA
tgttctattttaaagtacaggtaatcatgcatgagaagtcaaaacctttaaaactgtcaaacagtgggctgctgtgtgtggcatttgctgccaaccatgacaacctaagttcaacttaagagcccaacaatggaaaaagaccccttcaagttgtcctctgccatctacacatacaccaaagcaggacacaggtatgtacagaattcataacttcgtataatgtatgctatacgaagttatgttcgaacgaagttcctattctctagaaagtataggaacttcgctagactagtacgcgtgtacaccttgtaattgctgctctgagcaagttgccattttttctttttagaggttttcagtcatagcagtaatgctagttctggtttgagtggctgagcctgttgctaggggaaaaaagtatggatttaaacataaatcaataaaataattgtctttaatttcttcttaggacaagatctaatttgaaatattaaaagtggatacaaaactgtttccgaaatgcagacaattaagtgtgttgttgttggtgatggtgctgttggtaaaacatgtctcctgatatcctacacaacaaacaaattcccatcggaatatgtaccaactgtaagtataaaggctttttactagcaaaagattgtaatgtagtgtctgtccattggaaaacacttggcctgcctgcagtatttttgactgtcttgccctttaaaaaaaattaaattttactacctttattactttgtggggtgtgtgttataacttcgtataatgtatgctatacgaagttatggtaccgaattcagtttctggaccttgttgttttgtcttaagtatcaaagtagaacagtgaccgatatattccttttatttttttttttcttccctgagactgggtttctctgtgtagcccttgctgttctgtaactcactctgtgagtggcctcaaactcagagatccgcctgccttgggcaaggaaggtgctataaaaagagtctcgtgtggtatatgaagtatagtttgtgaaagctgcttcagtgtgagcacacacgcattatatgcaagaccaattgcagcccgaagaatactctaaaaaatgactcactgcccag

SEQ ID NO: 134 - DNA, 561 bp, synthetic construct
  misc_feature 1..561: note = Synthetic polynucleotide
  source 1..561: mol_type = other DNA
tgttctattttaaagtacaggtaatcatgcatgagaagtcaaaacctttaaaactgtcaaacagtgggctgctgtgtgtggcatttgctgccaaccatgacaacctaagttcaacttaagagcccaacaatggaaaaagaccccttcaagttgtcctctgccatctacacatacaccaaagcaggacacaggtatgtacagaattcataacttcgtataatgtatgctatacgaagttatggtaccgaattcagtttctggaccttgttgttttgtcttaagtatcaaagtagaacagtgaccgatatattccttttatttttttttttcttccctgagactgggtttctctgtgtagcccttgctgttctgtaactcactctgtgagtggcctcaaactcagagatccgcctgccttgggcaaggaaggtgctataaaaagagtctcgtgtggtatatgaagtatagtttgtgaaagctgcttcagtgtgagcacacacgcattatatgcaagaccaattgcagcccgaagaatactctaaaaaatgactcactgcccag

SEQ ID NO: 135 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 136 - protein, 16 aa, synthetic construct
  REGION 1..16: note = Synthetic polypeptide
HGYLLYDGYYEWYFDY